(19) United States
(12) Patent Application Publication
(10) Pub. No.: US 2007/0218117 A1
(43) Pub. Date: Sep. 20, 2007

(54) Supra molecular construct for delivery of interferon to a mammal

(75) Inventors: John R. Lau, Howard, OH (US); W. Blair Geho, Wooster, OH (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: SDG, Inc.

(21) Appl. No.: 11/384,575

(22) Filed: Mar. 20, 2006

(51) Int. Cl. A61K 38/21; A61K 9/127; A61K 31/717

(52) U.S. Cl. 424/450; 424/85.7; 514/57

(57) ABSTRACT

The instant invention is drawn to a hepatocyte targeted composition comprising interferon associated with a supra-molecular lipid construct comprising amphipathic lipid molecules and receptor binding molecule. The composition can comprise a mixture of free interferon and interferon associated with the complex. The composition can be modified to protect interferon and the complex from degradation. The invention also includes methods for the manufacture of the composition and loading interferon into the composition and recycling various components of the composition. Methods of treating individuals infected with the hepatitis C and other hepatitis viruses.

### BACKGROUND OF THE INVENTION

Hepatitis C virus (HCV) infection is the most common chronic bloodbome infection in the United States. The Center for Disease Control (CDC) estimates that during the 1980s, an average of 240,000 new infections occurred each year. Since the 1980's the number of new infections per year has declined to about 30,000 in 2003. It is estimated that approximately 3.9 million Americans, roughly 1.8% of the U.S. population, has been infected with HCV. Approximately 2.7 million of these people are chronically infected and might not be aware of their infection because they are not clinically ill. Infected persons serve as a source of transmission to others and are at risk for chronic liver disease or other HCV-related chronic diseases during the first two or more decades following initial infection.

Current treatment protocols for hepatitis C are based on the use of various preparations of interferon-alpha, which are administered by intramuscular or subcutaneous injection. Interferon alpha is a naturally occurring glycoprotein secreted by cells in response to viral infections. Interferon-alpha, which has immunomodulatory, antiproliferative and antiviral properties, exerts its effects by binding to a membrane receptor. Interferon-alpha plays a critical role in maintaining the balance of the immune system, and is produced normally by the body in very low concentrations compared to traditional injectable interferon therapy, which requires administration of high doses to achieve the concentrations needed at the disease site. If interferon alpha is administered directly into the bloodstream, very high doses—millions of international units (IU)—are required to assure that sufficient amounts reach the diseased tissue. Released interferon-alpha reaches a wide range of systems within the body rather than being delivered to targeted areas of the body. What is needed is a composition of interferon-alpha where interferon-alpha is released at a relatively constant rate over an extended time period and a portion of the interferon-alpha in the composition is targeted for delivery to the liver to better reduce or eliminate the hepatitis C virus.

Interferon alfa-2a (ROFERON-A®; Hoffmann-La Roche), interferon alpha-2b (INTRON-A®; Schering-Plough) and interferon alfacon-1 (INFERGEN®; Intermune) are approved in the United States for the treatment of adults with chronic hepatitis C as single agents. The recommended dose of interferons alfa-2b and alpha-2a for the treatment of chronic hepatitis C is 3,000,000 units three times a week, administered by subcutaneous or intramuscular injection. Treatment is administered for six months to two years. For interferon alfacon-1, the recommended dose is 9 micrograms three times a week for first time treatment and 15 micrograms three times a week for another six months for patients who do not respond or relapse. Treatment with interferon alone leads to a sustained response in less than 15% of subjects. Ribavirin, a synthetic nucleoside that has activity against a broad spectrum of viruses, is often administered in combination with interferon-alpha in the treatment of chronic hepatitis C.

Recently, peginterferon-alpha, sometimes called pegylated interferon, has been used for the treatment of chronic hepatitis C. Two preparations of peginterferon-alpha have been studied in patients with hepatitis C: peginterferon-alpha-2b (PEG-INTRON®; Schering-Plough) and peginterferon-alpha-2a (PEGASYS®; Hoffmann-La Roche). Peginterferon-alphas differ from unmodified interferon-alphas in that a polyethylene glycol molecule is attached to the interferon molecule. This structural modification results in a slower elimination from the body thereby higher, more constant blood levels of interferon-alpha are achieved with less frequent dosing. In contrast to unmodified interferon-alpha, which must be injected three times a week to treat chronic hepatitis C, peginterferon-alpha needs to be injected only once a week.

The main goal of treatment of chronic hepatitis C is to eliminate detectable viral RNA from the blood. Lack of detectable hepatitis C virus RNA from blood six months after completing therapy is known as a sustained response.

There is, therefore, an unmet need in the art for compositions and methods of treating patients infected with the hepatitis C virus. The present invention meets these needs by providing a long-acting composition that is targeted for delivery to the liver.

### BRIEF SUMMARY OF THE INVENTION

In one aspect, the present invention includes an interferon binding supra-molecular lipid construct comprising amphipathic lipid molecules and an extended amphipathic lipid, wherein the extended amphipathic lipid molecule comprises proximal, medial and distal moieties, wherein the proximal moiety connects the extended lipid molecule to the construct, the distal moiety binds the construct to a hepatocyte binding receptor in the liver, and the medial moiety connects the proximal and distal moieties.

In another aspect, the construct further comprises at least one interferon selected from the group consisting of interferon-alpha, interferon-alpha-2a, interferon-alpha-2b, interferon-alpha-n1, interferon-alpha-n3, peginterferon alpha 2a, peginterferon alpha 2b, a derivative thereof, or a combination of any of the aforementioned interferons.

In still another aspect, the construct further comprises an insoluble form of interferon associated with the supra-molecular lipid construct.

In another aspect, the construct further comprises at least one antiviral agent.

In yet another aspect, the amphipathic lipid molecules of the construct comprise at least one compound selected from the group consisting of 1,2-distearoyl-sn-glycero-3-phosphocholine, cholesterol, dicetyl phosphate, 1,2-dipalmitoyl-sn-glycerol-[3-phospho-rac-(1-glycero)], 1,2-distearoyl-sn-glycero-3-phosphoethanolamine, 1,2-dipalmitoyl-sn-glycero-3-phosphoethanolamine-N-(succinyl), derivatives thereof and mixtures of any of the foregoing compounds.

In one aspect, the construct further comprises interferon associated with a water insoluble target molecule complex, wherein the complex comprises multiple linked individual units, the multiple linked individual units comprise: a bridging component selected from the group comprising a transition element, an inner transition element, a neighbor element of the transition element and a mixture of any of the foregoing elements, and a complexing component, provided that when the transition element is chromium, a chromium target molecule complex is created, further wherein the multiple linked individual units are combined with the supra-molecular lipid construct matrix.

In another aspect, the construct comprises free interferon not associated with the target molecule complex.

In still another aspect, the bridging component is chromium.

In yet another aspect, the complexing component comprises poly(bis)-[(N-(2,6-diisopropylphenyl)carbamoyl methyl) iminodiacetic acid].

In one aspect, the proximal moiety of the extended amphipathic lipid comprises at least one, but not more than two, long acyl hydrocarbon chains bound to a glycerol backbone, wherein the hydrocarbon chains may be saturated, unsaturated or a mixture thereof.

In another aspect, the medial moiety of the extended amphipathic lipid comprises a thio-acetyl triglycine polymer or a derivative thereof, wherein the amphipathic lipid molecule extends from the surface of the interferon binding supra-molecular lipid construct.

In yet another aspect, the distal component of the extended amphipathic lipid comprises a non-polar derivatized benzene ring or a heterobicyclic ring structure.

In a further aspect, the construct comprises a positive charge or a negative charge or combinations thereof.

In one aspect, the extended amphipathic lipid molecule comprises at least one carbonyl moiety positioned at a distance approximately less than or equal to 13.5 angstroms from the terminal end of the distal moiety.

In another aspect, the extended amphipathic lipid molecule comprises at least one carbamoyl moiety comprising a secondary amine.

In still another aspect, the extended amphipathic lipid molecule comprises positively charged chromium in the medial position.

In one aspect, the construct further comprises cellulose acetate hydrogen phthalate.

In one aspect, the present invention includes a method of manufacturing an interferon binding supra-molecular lipid construct comprising: creating a mixture of the individual components of the lipid construct comprising amphipathic lipid molecules and an extended amphipathic lipid, and forming a suspension of the target molecule complex in water.

In yet another aspect, interferon is loaded into the supra-molecular lipid construct.

In one aspect, the loading of interferon comprises equilibrium and non-equilibrium loading.

In another aspect, loading interferon into the supra-molecular lipid construct comprises adding interferon to a mixture of the supra-molecular lipid construct in water and allowing the interferon to remain in contact with the mixture until equilibrium to be reached.

In still another aspect, interferon is terminally loaded into the supra-molecular lipid construct following the mixture reaching equilibrium, wherein the solution containing free interferon is removed from the construct, wherein the construct contains interferon bound to the construct.

In yet another aspect, a chromium complex comprising multiple linked individual units is added to the supra-molecular lipid construct.

In one aspect, the present invention includes adding cellulose acetate hydrogen phthalate to the supra-molecular lipid construct containing bound interferon.

In another aspect, a method of increasing the bioavailability of interferon in a patient comprises placing interferon within a supra-molecular lipid construct, wherein the supra-molecular lipid construct contains a plurality of non-covalent multi-dentate binding sites, the construct reducing the reactions of acids and enzymes in the stomach with the interferon and administering interferon to the patient.

In still another aspect, the supra-molecular lipid construct comprises interferon, 1,2-distearoyl-sn-glycero-3-phophocholine, cholesterol, dicetyl phosphate, 1,2-dipalmitoyl-sn-glycero-[3-phospho-rac-(1-glycerol)], 1,2-distearoyl-sn-glycero-3 -phosphoethanolamine, and 1,2-dipalmitoyl-sn-glycero-3-phosphoethanolamine-N-(succinyl) or derivatives, and a hepatocyte receptor binding molecule.

In yet another aspect, a method of treating a patient infected with hepatitis comprises administering to the patient an effective amount of a supra-molecular lipid construct comprising interferon associated with the construct.

In one aspect, the hepatitis comprises at least one hepatitis selected from the group consisting of Hepatitis B, Hepatitis C, Hepatitis D, Hepatitis E, Hepatitis F and Hepatitis G.

In another aspect, construct further comprises free interferon not associated with the target molecule complex.

In still another aspect, construct further comprises a target molecule complex, wherein the complex comprises multiple linked individual units.

In yet another aspect, the treatment is administered oral or subcutaneous.

In one aspect, the present invention includes a method for increasing the delivery of interferon to hepatocytes in the liver of a patient infected with hepatitis by administering to the patient a supra-molecular lipid construct comprising interferon and an extended lipid molecule comprising a moiety that binds to hepatocyte receptors, wherein the supra-molecular lipid construct is present in a plurality of sizes, wherein hepatocyte receptors bind optimally sized constructs to augment endocytosis and elicit the intended pharmacological action of the supra-molecular lipid construct.

In another aspect, interferon molecule within the supra-molecular lipid construct is protected from hydrolytic degradation by providing a shield of lipid molecules arranged in a three-dimensional structural array that prevents access by hydrolytic enzymes.

In still another aspect, cellulose acetate hydrogen phthalate is added to the supra-molecular lipid construct to react with individual lipid molecules.

In yet another aspect, an insolubilized dosage form of interferon is produced within the supra-molecular lipid construct.

In one aspect, the present invention includes a kit for treating hepatitis in a mammal, the kit comprising interferon and interferon binding supra-molecular lipid construct, the kit further comprising physiological buffer solution, an applicator, and an instructional material for the use thereof.

In another aspect, the kit is for treating hepatitis selected from the group consisting of Hepatitis B, Hepatitis C, Hepatitis D, Hepatitis E, Hepatitis F and Hepatitis G.

### BRIEF DESCRIPTION OF THE DRAWINGS

For the purposes of illustrating the invention, there is depicted in the drawings certain embodiments of the invention. However, the invention is not limited to the precise arrangements and instrumentalities of the embodiments depicted in the drawings.

FIG. 1 is a depiction of an interferon binding supra-molecular lipid construct comprising interferon, amphipathic lipid molecules and an extended amphipathic lipid.

FIG. 2 is depiction of a route for manufacturing biocytin.

FIG. 3 is a depiction of a route for manufacturing iminobiocytin.

FIG. 4 is a depiction of a route for manufacturing benzoyl thioacetyl triglycine iminobiocytin (BTA-3gly-iminobiocytin).

FIG. 5 is a depiction of a route for manufacturing benzoyl thioacetyl triglycine.

FIG. 6 is a depiction of a route for manufacturing benzoyl thioacetyl triglycine sulfo-N-hydroxysiccinimide (BTA-3-gly-sulfo-NHS).

FIG. 7 is a depiction of a route for manufacturing benzoyl thioacetyl triglycine iminobiocytin (BTA-3-gly-iminobiocytin).

FIG. 8 is a depiction of a route for manufacturing a lipid anchoring and hepatocyte receptor binding molecule (LA-HRBM).

FIG. 9 is a depiction of the change in structure of iminobiotin under acidic versus basic conditions.

FIG. 10 is a depiction of potential sites for binding between cellulose acetate hydrogen phthalate and interferon.

FIG. 11 is an outline of a method of manufacturing an interferon binding supra-molecular lipid construct comprising amphipathic lipid molecules and an extended amphipathic lipid.

FIG. 12 is comprised of two parts. FIG. 12a indicates the relative expression level in the liver and spleen from mice dosed with interferon alpha. FIG. 12b indicates the relative expression level in the liver and spleen from mice dosed with interferon alpha plus HDV.

FIG. 13 indicates the effect of HDV targeting on hepatic PKR activation by interferon alpha in a mouse model.

### DETAILED DESCRIPTION OF THE INVENTION

This invention includes a supra-molecular lipid construct comprising interferon, amphipathic lipid molecules and an extended amphipathic lipid (a receptor binding molecule), wherein the extended amphipathic lipid molecule comprises proximal, medial and distal moieties, wherein the proximal moiety connects the extended lipid molecule to the construct, the distal moiety connects the construct to a hepatocyte binding receptor in the liver, and the medial moiety connects the proximal and distal moieties. A supra-molecular lipid construct is a spherical lipid and phospholipid particle in which individual lipid molecules cooperatively interact to create a bipolar lipid membrane which encloses and isolates a portion of the medium in which it was formed. The supra-molecular lipid construct can target the delivery of interferon to the hepatocytes in the liver and provide for a sustained release of interferon to better reduce or eliminate the hepatitis C virus. This invention includes a hepatocyte targeted pharmaceutical composition where interferon is associated with a water insoluble target molecule complex within the construct and the composition is targeted to hepatocytes in the liver of a patient to provide an effective means of managing Hepatitis C virus.

The composition can be administered by various routes, including subcutaneously or orally, for the purpose of treating mammals infected with the hepatitis C virus.

The invention further provides a method of manufacturing a supra-molecular lipid construct comprising interferon, amphipathic lipid molecules and an extended amphipathic lipid, wherein the extended amphipathic lipid molecule comprises proximal, medial and distal moieties, wherein the proximal moiety connects the extended lipid molecule to the construct, the distal moiety connects the construct to a hepatocyte binding receptor in the liver, and the medial moiety connects the proximal and distal moieties. The invention also provides a method of manufacturing a composition comprising free interferon and interferon associated with a water insoluble target molecule complex within the construct that targets delivery of the complex to hepatocytes. The target molecule complex is composed of multiple linked individual units of a structure formed by a metal complex contained within a supra-molecular lipid construct matrix.

Additionally, the invention provides methods of treating individuals infected with hepatitis C by administering an effective dose of a supra-molecular lipid construct comprising interferon, amphipathic lipid molecules and an extended amphipathic lipid, targeted for delivery to hepatocytes. The invention also provides methods of treating individuals infected with hepatitis C by administering an effective dose of a supra-molecular lipid construct comprising interferon, amphipathic lipid molecules, an extended amphipathic lipid and a water insoluble target molecule complex, targeted for delivery to hepatocytes.

Definitions

Unless defined otherwise, all technical and scientific terms used herein generally have the same meaning as commonly understood by one of ordinary skill in the art to which this invention belongs. Generally, the nomenclature used herein and the laboratory procedures in organic chemistry and protein chemistry are those well known and commonly employed in the art.

The articles “a” and “an” are used herein to refer to one or to more than one (i.e., to at least one) of the grammatical object of the article. By way of example, “an element” means one element or more than one element.

The term “active ingredient” refers to interferon and other pharmacologically active compounds.

As used herein, amino acids are represented by the full name thereof, by the three letter code corresponding thereto, as indicated in the following table:

| Full Name | Three-Letter Code |
|---|---|
| Alanine | Ala |
| Arginine | Arg |
| Asparagine | Asn |
| Aspartic Acid | Asp |
| Cysteine | Cys |
| Cystine | Cys-Cys |
| Glutamic Acid | Glu |
| Glutamine | Gln |
| Glycine | Gly |
| Histidine | His |
| Isoleucine | Ile |
| Leucine | Leu |
| Lysine | Lys |
| Methionine | Met |
| Phenylalanine | Phe |
| Proline | Pro |
| Serine | Ser |
| Threonine | Thr |
| Tryptophan | Trp |
| Tyrosine | Tyr |
| Valine | Val |

The term “lower” means the group it is describing contains from 1 to 6 carbon atoms.

The term “alkyl”, by itself or as part of another substituent means, unless otherwise stated, a straight, branched or cyclic chain hydrocarbon having the number of carbon atoms designated (i.e. C₁-C₆ means one to six carbons) and includes straight, branched chain or cyclic groups. Examples include: methyl, ethyl, propyl, isopropyl, butyl, isobutyl, tert-butyl, pentyl, neopentyl, hexyl, cyclohexyl and cyclopropylmethyl. Most preferred is (C₁-C₃) alkyl, particularly ethyl, methyl and isopropyl.

The term “alkylene”, by itself or as part of another substituent means, unless otherwise stated, a straight, branched or cyclic chain hydrocarbon having two substitution sites, e. g., methylene (—CH₂—), ethylene (—CH₂CH₂—), isopropylene (—CH(CH₃)═CH₂), etc.

The term “aryl”, employed alone or in combination with other terms, means, unless otherwise stated, a cyclic carbon ring structure, with or without saturation, containing one or more rings (typically one, two or three rings) wherein such rings may be attached together in a pendant manner, such as a biphenyl, or may be fused, such as naphthalene. Examples include phenyl; anthracyl; and naphthyl. The structure can have one or more substitution sites where functional groups, such as alcohol, alkoxy, amides, amino, cyanides, halogen, and nitro, are bound.

The term “arylloweralkyl” means a functional group wherein an aryl group is attached to a lower alkylene group, e.g., —CH₂CH₂-phenyl.

The term “alkoxy” employed alone or in combination with other terms means, unless otherwise stated, an alkyl group or an alkyl group containing a substituent such as a hydroxyl group, having the designated number of carbon atoms connected to the rest of the molecule via an oxygen atom, such as, for example, —OCHOH—, —OCH₂OH, methoxy (—OCH₃), ethoxy (—OCH₂CH₃), 1-propoxy (—OCH₂CH₂CH₃), 2-propoxy (isopropoxy), butoxy (—OCH₂CH₂CH₂CH₃), pentoxy (—OCH₂CH₂CH₂CH₂CH₃), and the higher homologs and isomers.

The term “acyl” means a functional group of the general formula —C(═O)—R, wherein —R is hydrogen, hydrocarbyl, amino or alkoxy. Examples include acetyl (—C(═O)CH₃), propionyl (—C(═O)CH₂CH₃), benzoyl (—C(═O)C₆H₅), phenylacetyl (—C(═O)CH₂C₆H₅), carboethoxy (—CO₂ CH₂CH₃), and dimethylcarbamoyl (—C(═O)N(CH₃)₂).

The terms “halo” or “halogen” by themselves or as part of another substituent mean, unless otherwise stated, a fluorine, chlorine, bromine, or iodine atom.

The term “heterocycle” or “heterocyclyl” or “heterocyclic” by itself or as part of another substituent means, unless otherwise stated, an unsubstituted or substituted, stable, mono- or multicyclic heterocyclic ring system comprising carbon atoms and at least one heteroatom selected from the group comprising N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure. Examples include pyrrole, imidazole, benzimidazole, phthalein, pyridenyl, pyranyl, furanyl, thiazole, thiophene, oxazole, pyrazole, 3-pyrroline, pyrrolidene, pyrimidine, purine, quinoline, isoquinoline, carbazole, etc.

The term “chromium target molecule complex” refers to a complex comprising a number of individual units, where each unit comprises chromium (Cr) atoms capable of accepting up to six ligands contributed by multivalent molecules, such as ligands from numerous molecules of N-(2,6-diisopropylphenylcarbamoylmethyl) iminodiacetic acid. The individual units are linked to each other forming a complicated polymeric structure linked in a three-dimensional array. The polymeric complex is insoluble in water but soluble in organic solvents.

The term “supra-molecular lipid construct” refers to a spherical lipid and/or phospholipid particle in which individual lipid molecules cooperatively interact to create a bipolar lipid membrane which encloses and isolates a portion of the medium in which it was formed.

The term “amphipathic lipid molecule” means a lipid molecule having a polar and non-polar end.

The term “extended amphipathic lipid” means an amphipathic molecule with a structure that, when part of a supra-molecular construct, extends from the supra-molecular construct into media around the construct, and can attach to a receptor.

A “complexing agent” is a compound that will form a polymeric complex with a selected metal bridging agent, e. g. a salt of chromium, zirconium, etc., that exhibits polymeric properties where the polymeric complex is substantially insoluble in water and soluble in organic solvents.

By “substantially soluble” is meant that the polymeric complex, such as the resultant polymeric chromium target molecule complex or other metal targeting complexes which may be crystalline or amorphous in composition that are formed from complexing agents, exhibit the property of being insoluble in water at room temperature. Such a polymeric complex or a dissociated form thereof when associated with a supra-molecular lipid construct matrix forms a transport agent which functions to carry and deliver interferon to hepatocytes in the liver of a warm-blooded host.

By use of the term “associated with” is meant that the referenced material is incorporated into or on the surface of, or within, the supra-molecular lipid construct matrix.

The term “interferon” refers to natural or recombinant forms of interferon, including the alpha, beta, gamma and other forms of interferon, peginterferons and derivatives of the aforementioned interferons.

“HDV”, or “Hepatocyte Delivery Vehicle”, is a water insoluble target molecule complex comprising a supra-molecular lipid construct matrix containing multiple linked individual units of a structure formed by the combination of a metal bridging agent and a complexing agent. “HDV” is described in WO 99/59545, Targeted Liposomal Drug Delivery System.

“Statistical structure” denotes a structure formed from molecules that can migrate from one supra-molecular construct to another and the structure is present in a plurality of particle sizes that can be represented by a Gaussian distribution.

“Multi-dentate binding” is a chemical binding process that utilizes multiple binding sites within the supra-molecular lipid construct, such as cellulose acetate hydrogen phthalate, phospholipids and interferon. These binding sites promote hydrogen bonding, ion-dipole and dipole-dipole interactions where the individual molecules work in tandem to form non-covalent associations that serve to bind or connect two or more molecules.

As used herein, to “treat” means reducing the frequency with which symptoms of a disease, disorder, or adverse condition, and the like, are experienced by a patient.

As used herein, the term “pharmaceutically acceptable carrier” means a chemical composition with which the active ingredient may be combined and which, following the combination, can be used to administer the active ingredient to a subject.

As used herein, the term “physiologically acceptable” means that the ingredient is not deleterious to the subject to which the composition is to be administered.

Description of the Invention—Composition

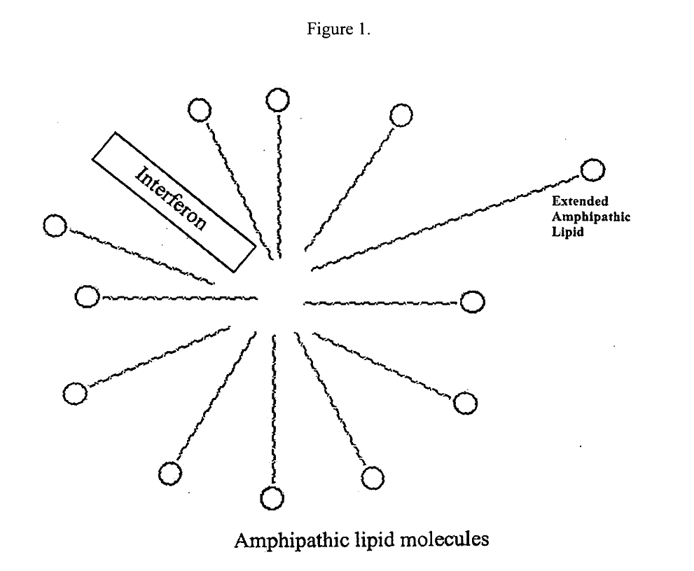

A depiction of an interferon binding supra-molecular lipid construct comprising interferon, amphipathic lipid molecules and an extended amphipathic lipid is shown in FIG. 1. The extended amphipathic lipid molecule, also known as a receptor binding molecule, comprises proximal, medial and distal moieties, wherein the proximal moiety connects the extended lipid molecule to the construct, the distal moiety connects the construct to a hepatocyte binding receptor in the liver, and the medial moiety connects the proximal and distal moieties. Suitable amphipathic lipid molecules generally comprise a polar head group and non-polar tail group that are attached to each other through a glycerol-backbone.

Suitable amphipathic lipid molecules include 1,2-distearoyl-sn-glycero-3-phosphocholine, 1,2-dipalmitoyl-sn-glycero-3-phosphocholine, 1,2-dimyristoyl-sn-glycero-3-phosphocholine, cholesterol, cholesterol oleate, dicetyl phosphate, 1,2-distearoyl-sn-glycero-3-phosphate, 1,2-dipalmitoyl-sn-glycero-3-phosphate, 1,2-dimyristoyl-sn-glycero-3-phosphate, 1,2-dipalmitoyl-sn-glycero-3-phosphoethanolamine-Cap Biotinyl), 1,2-distearoyl-sn-glycero-3-phosphoethanolamine, 1,2-dipalmitoyl-sn-glycero-3-phosphoethanolamine-N-(succinyl), 1,2-dipalmitoyl-sn-glycero-3-[phospho-rac-(1-glycerol)] (sodium salt), and a mixture of any of the foregoing lipids or appropriate derivative of these lipids which are given in Table 1.

TABLE 1

| | | |
|---|---|---|
| 1 | 1,2-distearoyl-sn- glycero-3- phosphocholine | 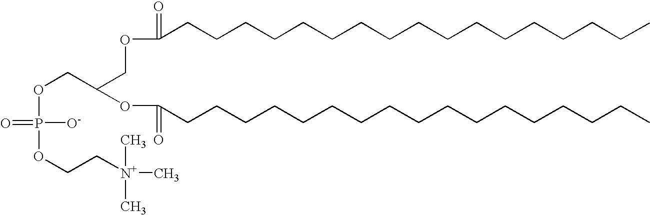 |
| 2 | 1,2-dipalmitoyl-sn- glycero-3- phosphocholine | 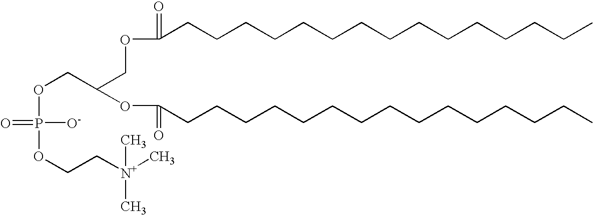 |
| 3 | 1,2-dimyristoyl-sn- glycero-3- phosphocholine | 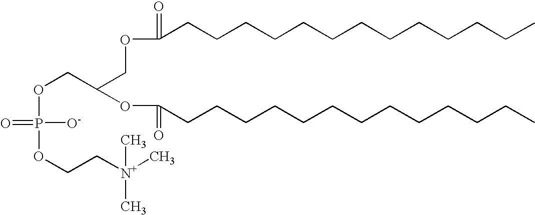 |
| 4 | Cholesterol | 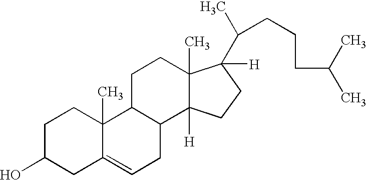 |

In an embodiment, amphipathic lipid molecules include 1,2-distearoyl-sn-glycero-3-phosphocholine, cholesterol, dicetyl phosphate, 1,2-dipalmitoyl-sn-glycero-3-phosphoethanolamine-N-(Cap Biotinyl); 1,2-distearoyl-sn-glycero-3-phosphoethanolamine, 1,2-dipalmitoyl-sn-glycero-3-phosphoethanolamine-N-(succinyl), 1,2-dipalmitoyl-sn-glycero-3-[phospho-rac-(1-glycerol)] (sodium salt) and a mixture of any of the foregoing lipids.

The extended amphipathic lipid molecule, also know as a receptor binding molecule, comprises proximal, medial and distal moieties. The proximal moiety connects the extended lipid molecule to the construct, and the distal moiety connects the construct to a hepatocyte binding receptor in the liver. The proximal and distal moieties are connected through a medial moiety. The composition of various receptor binding molecules is described below. Within a supra-molecular construct to, hepatocyte receptor binding molecules from one or more of the groups listed below can be present to bind the construct to receptors in the hepatocytes.

One group of hepatocyte receptor binding molecules comprises a terminal biotin or iminobiotin moiety, as well as derivatives thereof. The structural formulas of biotin, iminobiotin, carboxybiotin and biocytin are shown in Table 2. These molecules can be attached to a phospholipid molecule using a variety of techniques to create lipid anchoring molecules that can be intercalated into a supra-molecular lipid construct. These hepatocyte receptor binding molecules comprise an anchoring portion located in the proximal position to the supra-molecular lipid construct. The anchor portion comprises two lipophilic hydrocarbon chains that can associate and bind with other lipophilic hydrocarbon chains on phospholipid molecules within the supra-molecular lipid construct.

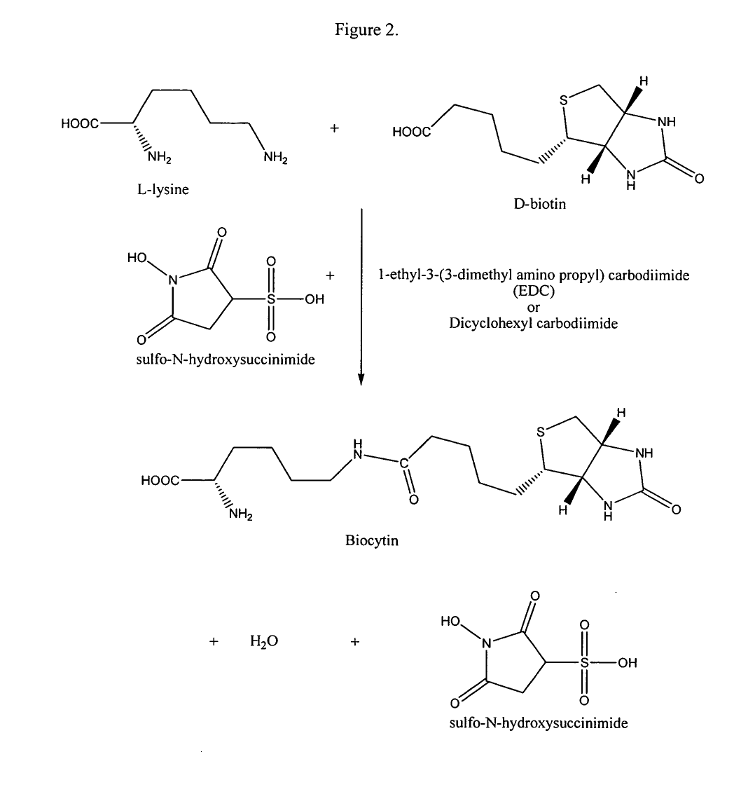

In a preferred embodiment, a second group of hepatocyte receptor binding molecules comprises a terminal biotin or iminobiotin moiety located in the distal position from the supra-molecular lipid construct. Both biotin and iminobiotin contain a mildly lipophilic bicyclic ring structure attached to a five-carbon valeric acid chain at the 4-carbon position on the bicyclic ring. In an embodiment, L-lysine amino acid may be covalently bound to the valeric acid C-terminal carboxyl functional group by reacting the carboxyl group on valeric acid with either the N-terminal α-amino group or the ε-amino group of L-lysine. This coupling reaction is performed using carbodiimide conjugation methods and results in the formation of an amide bond between L-lysine and biotin, as illustrated in FIG. 2.

TABLE 2

| | | |
|---|---|---|
| 1 | Biotin | 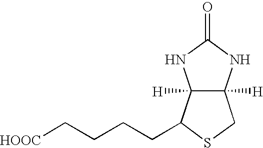 |
| 2 | Iminobiotin | 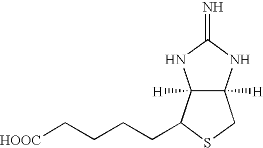 |
| 3 | Carboxybiotin | 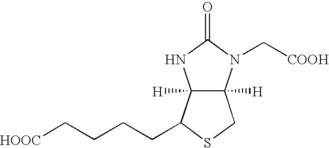 |
| 4 | Biocytin | 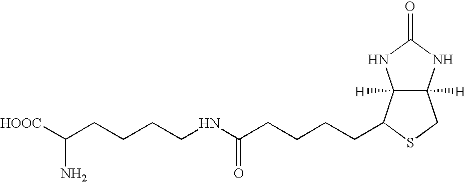 |

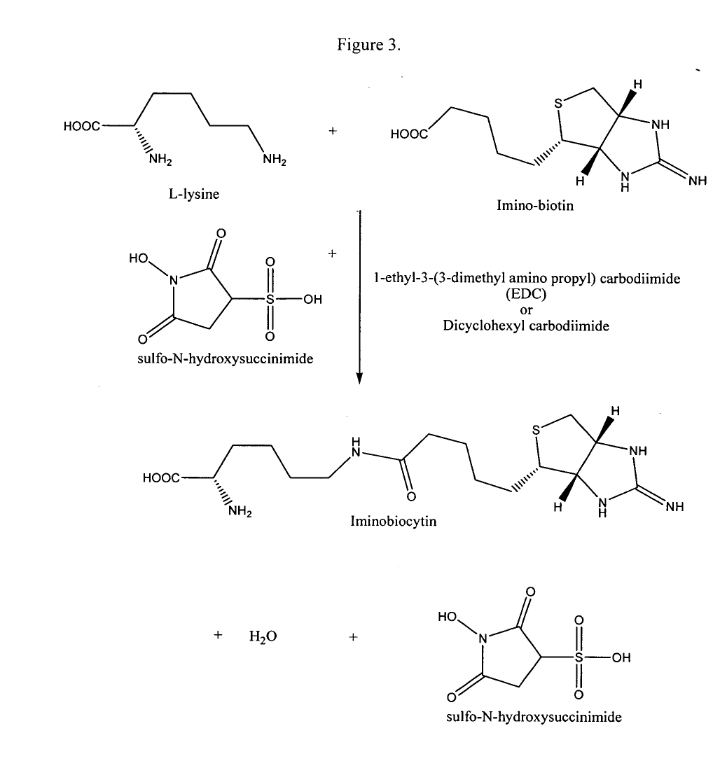

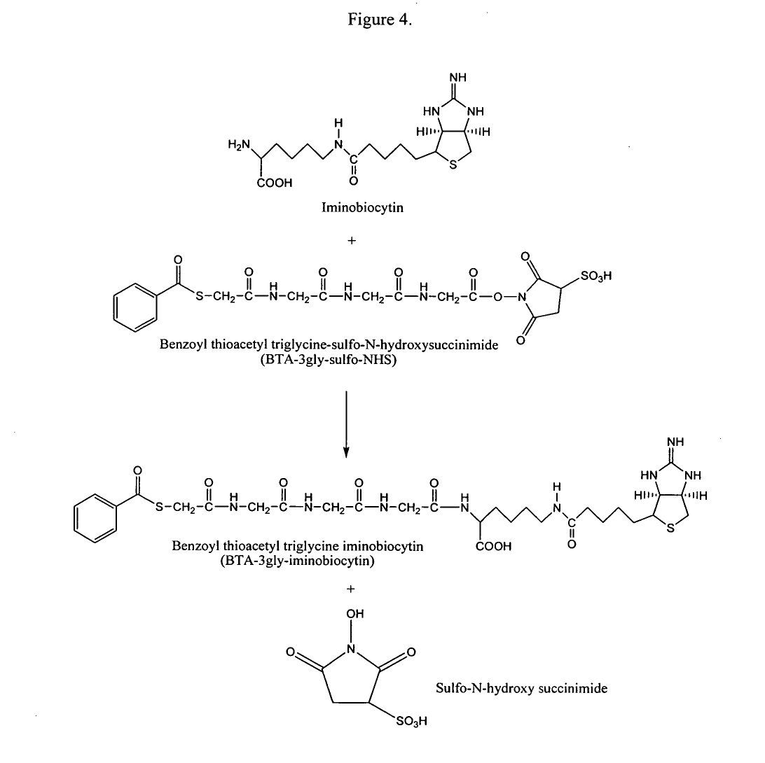

A third group of hepatocyte receptor binding molecules comprise iminobiotin, carboxybiotin and biocytin with the valeric acid side chain attached via an amide bond to either the α-amino group or the ε-amino group of the amino acid L-lysine. A preferred embodiment uses iminobiotin in forming an iminobiocytin moiety as shown in FIG. 3. During synthesis of the hepatocyte receptor binding molecule, the α-amino group of iminobiocytin can react with the activated ester benzoyl thioacetyl triglycine-N-hydroxysuccinimide (BTA-3gly-sulfo-NHS) to form the active hepatocyte binding molecule (BTA-3gly-iminobiocytin) as shown in FIG. 4. BTA-3gly-iminobiocytin functions as a molecular spacer that ultimately expresses an active nucleophilic sulfhydral functional group that can be used in subsequent coupling reactions. The spacer is located in the medial position in relation to the supra-molecular lipid construct and allows the terminal iminobiocytin moiety to extend approximately thirty angstroms from the surface of the supra-molecular construct to develop an optimal and non-restricted orientation of iminobiocytin for binding to the hepatocyte receptor. The medial spacer can include other derivatives that provide the correct stereo-chemical orientation for the terminal biotin moiety. The main function of the medial spacer is to properly and covalently connect the proximal and distal moieties in a linear array.

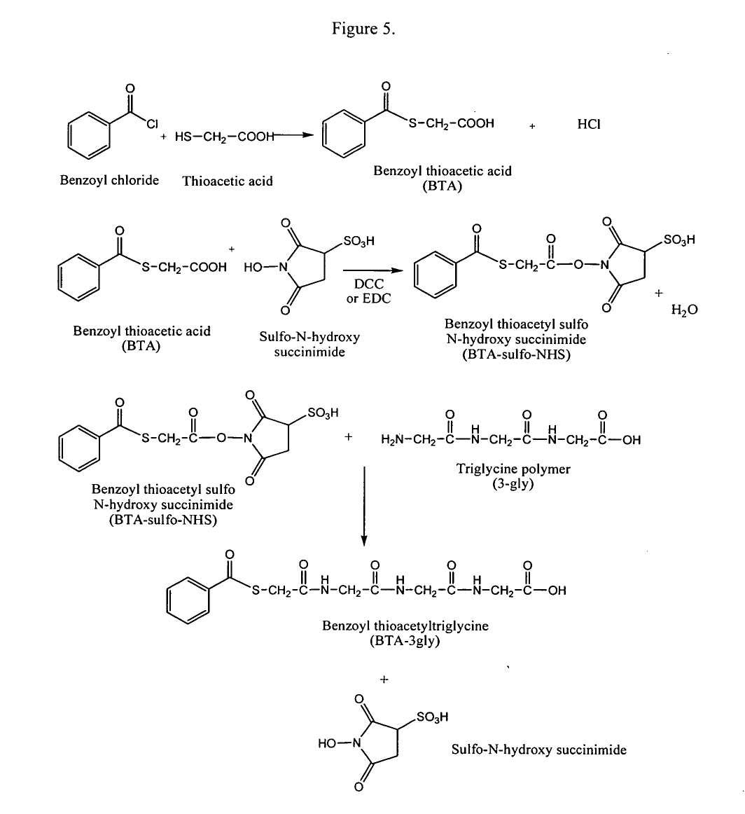

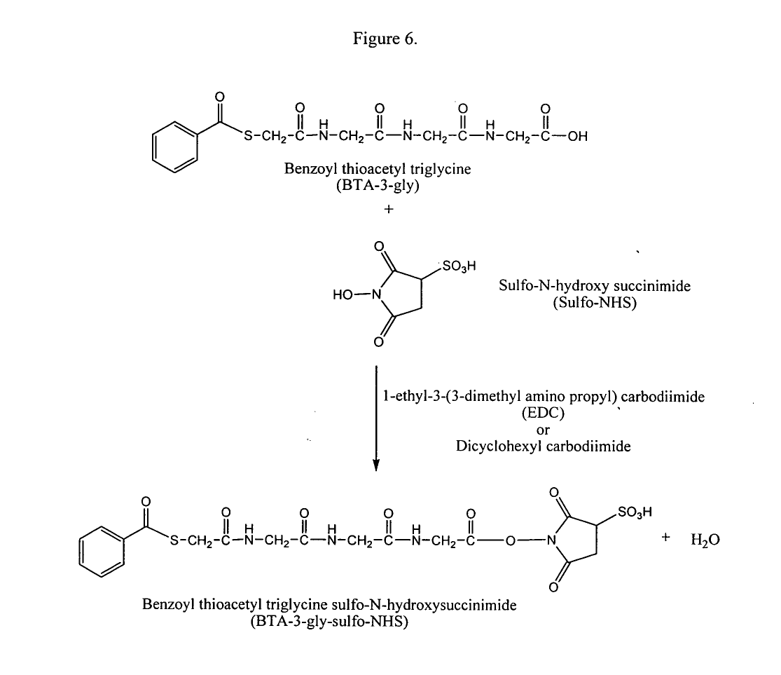

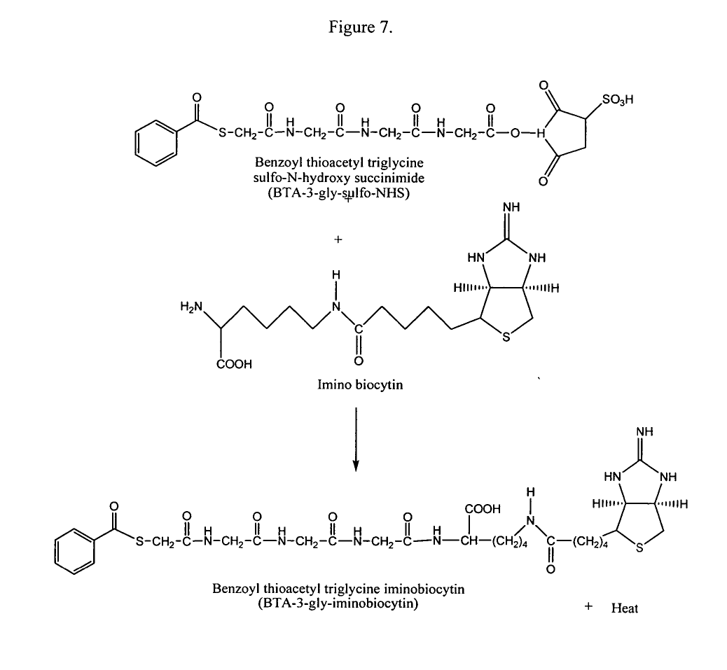

The BTA-3gly-sulfo-NHS portion of the hepatocyte receptor binding molecule can be synthesized by a number of means and in subsequent steps be linked to biocytin or iminobiocytin. The initial step comprises adding benzoyl chloride to thioacetic acid to form by nucleophilic addition a protective group for the active thio functionality. The products of the reaction are the benzoyl thioacetic acid complex and hydrochloric acid, as shown in FIG. 5. Additional steps in the synthesis involve reacting benzoyl thioacetic acid with sulfo-N-hydroxysuccinimide using dicyclohexylcarbodiimide or 1-ethyl-3-(3-dimethylaminopropyl) carbodiimide as a coupling agent to form benzoyl thioacetyl sulfo-N-hydroxysuccinimide (BTA-sulfo-NHS), as depicted in FIG. 5. Benzoyl thioacetyl sulfo-N-hydroxysuccinimide is then reacted with the amino acid polymer (glycine-glycine-glycine). Following nucleophilic attack by the α-amino group of triglycine, benzoyl thioacetyl triglycine (BTA-3gly) is formed while the sulfo-N-hydroxysuccinimide leaving group is solubilized by aqueous media, as shown in FIG. 5. Benzoyl thioacetyl triglycine is again reacted with dicyclohexylcarbodiimide or 1-ethyl-3-(3-dimethylaminopropyl) carbodiimide to form an ester bond with sulfo-N-hydroxysuccinimide, as shown in FIG. 6. The sulfo-N-hydroxysuccinimide ester of activated benzoyl thioacetyl triglycine (BTA-3gly-sulfo-NHS) is then reacted with the a-amino group of the L-lysine functionality of biocytin or iminobiocytin to form the hepatocyte receptor binding moiety, the extended amphipathic lipid molecule of benzoyl thioacetyl triglycine-iminobiocytin (BTA-3gly-iminobiocytin) illustrated in FIG. 7.

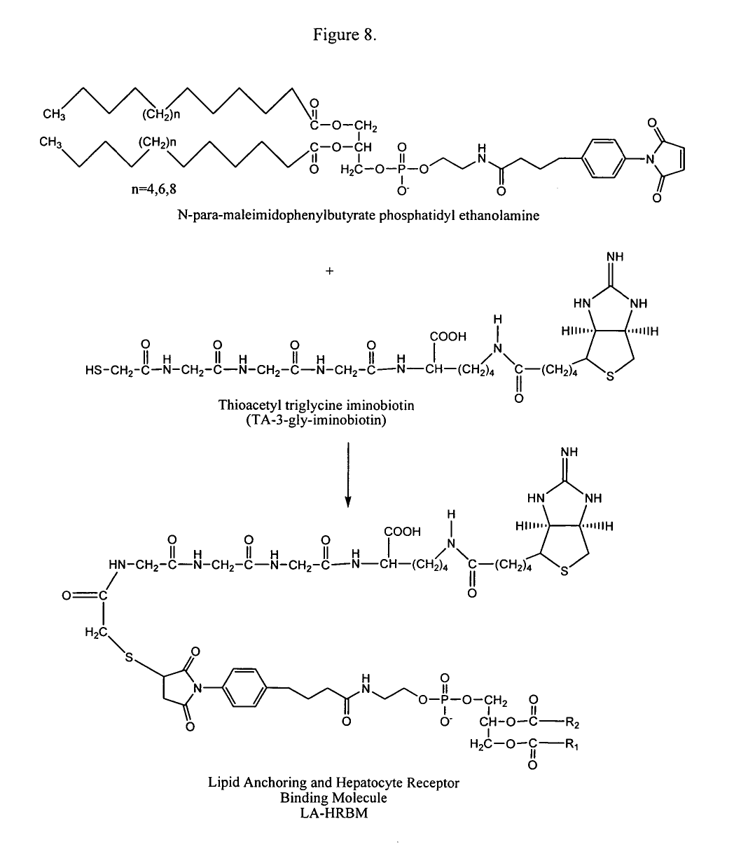

A second major coupling reaction for the synthesis of an hepatocyte receptor binding molecule is illustrated where benzoyl thioacetyl triglycine iminobiocytin is covalently attached through a thioether bond to a N-para-maleimidophenylbutyrate phosphatidylethanolamine, a preferred phospholipid anchoring molecule. This reaction results in a molecule that provides the correct molecular spacing between the terminal iminobiocytin ring and the supra-molecular lipid construct. An entire reaction scheme for forming a hepatocyte receptor binding molecule that functions as an extended amphipathic lipid molecule is depicted in FIG. 8. Prior to reacting benzoyl thioacetyl triglycine iminobiocytin with N-para-maleimidophenylbutyrate phosphatidylethanolamine to form a thioether linkage, the benzoyl protecting group is removed by heating in order to expose the free sulfhydral functionality. The reaction should be performed in an oxygen free environment to minimize oxidation of the sulfhydrals to the disulfide. Further oxidation could lead to the formation of a sulfone, sulfoxide, sulfenic acid or sulfonic acid derivative.

In an embodiment, the anchoring moiety of the molecule contains a pair of acyl hydrocarbon chains that form a lipid portion of the molecule. This portion of the molecule is non-covalently bound within the lipid domains of the supra-molecular lipid construct. In an embodiment the anchoring moiety is produced from is N-para-maleimidophenylbutyrate phosphatidylethanolamine. Other anchoring molecules may be used. In an embodiment, anchoring moleclules can include thio-cholesterol, cholesterol oleate, dicetyl phosphate; 1,2-distearoyl-sn-glycero-3-phosphoethanolamine, 1,2-dipalmitoyl-sn-glycero-3-phosphoethanolamine-N-(succinyl), 1,2-dipalmitoyl-sn-glycero-3-[phospho-rac-(1-glycerol)] (sodium salt), and mixtures, thereof. The entire molecular structure of the fully developed lipid anchoring and hepatocyte receptor binding molecule designated LA-HRBM is shown in FIG. 8.

A fourth group of hepatocyte receptor binding molecule comprises amphipathic organic molecules having both a water-soluble moiety and a water-insoluble moiety. The water-insoluble moiety reacts with a medial or connector moiety by coordination and bioconjugation chemical reactions, while the water-insoluble moiety binds to the hepatocyte binding receptor in the liver. The molecule contains a distal component comprising either by a non-polar derivatized benzene ring structure, such as a 2,6-diisopropylbenzene derivative, or by a lipophilic heterobicyclic ring structure. The entire hepatocyte receptor binding molecule possesses fixed or transient charges, either positive or negative, or various combinations thereof. These molecules contain at least one carbonyl group located equal to or less than, but not greater than, approximately 13.5 angstroms from the terminal end of the distal moiety, and at least one carbamoyl moiety containing a secondary amine and carbonyl group. The presence of a carbamoyl moiety or moieties enhances the molecular stability of the organic molecule. A plurality of secondary amines can be present within the molecule. These secondary amines contain a pair of unshared electrons allowing for ion-dipole and dipole-dipole bonding interactions with other molecules within the construct. These amines enhance molecular stability and provide a partially created negative charge that interacts with the distal moiety to promote hepatocyte receptor binding and specificity. An example of this group of receptor binding molecules is polychromium-poly(bis)-[N-(2,6-(diisopropylphenyl)carbamoyl methyl)imino diacetic acid]. In an embodiment, chromium III is located in the medial position of the hepatocyte receptor binding molecule. The proximal moiety of the hepatocyte specific binding molecule contains hydrophobic and/or non-polar structures that allow the molecules to be intercalated into, and subsequently bound within, the supra-molecular lipid construct. The medial and proximal moieties also allow for the correct stereo-chemical orientation of the distal portion of the hepatocyte receptor binding molecule.

The structure and properties of the supra-molecular lipid construct are governed by the structure of the lipids and interaction between lipids. The structure of the lipids is governed primarily by covalent bonding. Covalent bonding is the molecular bonding force necessary to retain the structural integrity of the molecules comprising the individual constituents of the supra-molecular lipid construct. Through non-covalent interactions between lipids, the lipid construct is maintained in a three-dimensional conformation.

The non-covalent bond can be represented in general terms by an ion-dipole dipole or induced ion-dipole bond, and by the hydrogen bonds associated with the various polar groups on the head of the lipid. Hydrophobic bonds and van der Waal's interactions can be generated through induced dipole associations between the lipid acyl chains. These bonding mechanisms are transient in nature and result in a bond-making and bond breaking process that occurs in a sub-femtosecond time interval. For example, van der Waal's interaction arises from a momentary change in dipole moment arising from a brief shift of orbital electrons to one side of one atom or molecule, creating a similar shift in adjacent atoms or molecules. The proton assumes a δ⁺ charge and the single electron a δ³¹ charge, thus forming a dipole. Dipole interactions occur with great frequency between the hydrocarbon acyl chains of amphipathic lipid molecules. Once individual dipoles are formed they can momentarily induce new dipole formation in neighboring atoms containing a methylenic (—CH₂—) functionality. A plurality of transiently induced dipole interactions are formed between acyl lipid chains throughout the supra-molecular lipid construct. These induced dipole interactions last for only a fraction of a femtosecond (1×10⁻¹⁵ sec) but exert a strong force when functioning collectively. These interactions are constantly changing and have a force approximately one-twentieth the strength of a covalent bond. They are nevertheless responsible for transient bonding between stable covalent molecules that determine the three-dimensional statistical structure of the construct and the stereo-specific molecular orientation of molecules within the supra-molecular lipid construct.

As a consequence of these induced-dipole interactions, the structure of the supra-molecular lipid construct is maintained by the exchange of lipid components between constructs. While the composition of the individual components of the construct is fixed, individual components of supra-molecular lipid constructs are subject to exchange reactions between constructs. These exchanges are initially governed by zero-order kinetics when a lipid component departs from a supra-molecular lipid construct. After the lipid component is released from the lipid construct, it may be recaptured by a neighboring supra-molecular lipid construct. The recapture of the released component is controlled by second-order reaction kinetics, which is affected by the concentration of the released component in aqueous media around the construct capturing the component and the concentration of the supra-molecular lipid construct which is capturing the released component.

Examples of extended amphipathic lipids, along with their respective identifiers, shown in Table 3, are: N-hydroxysuccinimide (NHS) biotin [1]; sulfo-NHS-biotin [2]; N-hydroxysuccinimide long chain biotin [3], sulfo-N-hydroxysuccinimide long chain biotin [4]; D-biotin [5]; biocytin [6]; sulfo-N-hydroxysuccinimide-S-S-biotin [7]; biotin-BMCC [8]; biotin-HPDP [9]; iodoacetyl-LC-biotin [10]; biotin-hydrazide [11]; biotin-LC-hydrazide [12]; biocytin hydrazide [13]; biotin cadaverine [14]; carboxybiotin [15]; photobiotin [16]; ρ-aminobenzoyl biocytin trifluoroacetate [17]; ρ-diazobenzoyl biocytin [18]; biotin DHPE [19]; biotin-X-DHPE [20 ]; 12-((biotinyl)amino)dodecanoic acid [21 ]; 12-((biotinyl)amino)dodecanoic acid succinimidyl ester [22]; S-biotinyl homocysteine [23]; biocytin-X [24]; biocytin x-hydrazide [25]; biotinethylenediamine [26]; biotin-XL [27]; biotin-X-ethylenediamine [28]; biotin-XX hydrazide [29]; biotin-XX-SE [30]; biotin-XX, SSE [31]; biotin-X-cadaverine [32]; α-(t-BOC)biocytin [33]; N-(biotinyl)-N′-(iodoacetyl)ethylenediamine [34]; DNP-X-biocytin-X-SE [35]; biotin-X-hydrazide [36]; norbiotinamine hydrochloride [37 ]; 3-(N-maleimidylpropionyl)biocytin [38]; ARP [39]; biotin-1-sulfoxide [40]; biotin methyl ester [41]; biotin-maleimide [42]; biotin-poly(ethyleneglycol)amine [43]; (+) biotin 4-amidobenzoic acid sodium salt [44]; Biotin 2-N-acetylamino-2-deoxy-β-D-glucopyranoside [45]; Biotin-α-D-N-acetylneuraminide [46]; Biotin-α-L-fucoside [47]; Biotin lacto-N-bioside [48]; Biotin-Lewis-A trisaccharide [49]; Biotin-Lewis-Y tetrasaccharide [50]; Biotin-α-D-mannopyranoside [51]; biotin 6-O-phospho-α-D-mannopyranoside [52]; and polychromium-poly(bis)-[N-(2,6-(diisopropylphenyl) carbamoyl methyl)imino]diacetic acid [53].

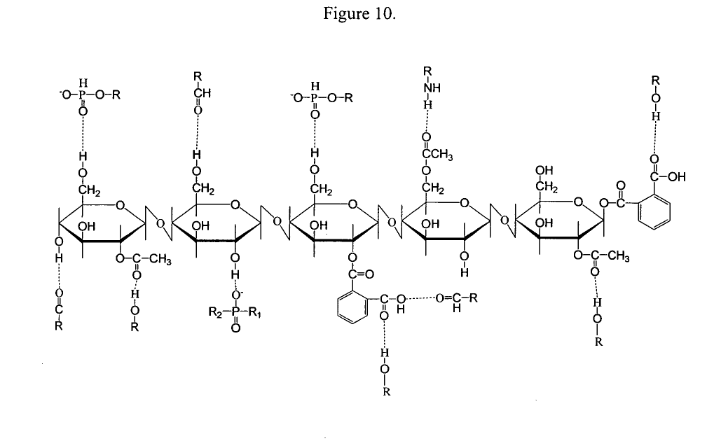

In an embodiment, a cellulose acetate hydrogen phthalate polymer is incorporated into the supra-molecular lipid construct where it can bind to hydrophilic functional groups on the interferon molecule and protect interferon from hydrolytic degradation. Cellulose acetate hydrogen phthalate comprises two glucose molecules linked beta (1→4) in a polymeric arrangement in which some of the hydrogen atoms on the hydroxyl groups of the polymer are replaced by an acetyl functionality (a methyl group bound to a carbonyl carbon) or a phthalate group (represented by a benzene ring with two carboxyl groups in the first and second positions of the benzene ring). The structural formula of cellulose acetate hydrogen phthalate polymer is shown in FIG. 10. Only one carboxyl group on the phthalate ring structure is involved in a covalent ester linkage to the cellulose acetate molecule. The other carboxyl group, which contains a

TABLE 3

| | | |
|---|---|---|
| 1 | N-hydroxysuccinimide (NHS) biotin | 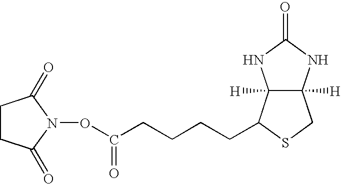 |
| 2 | sulfo-NHS-biotin | 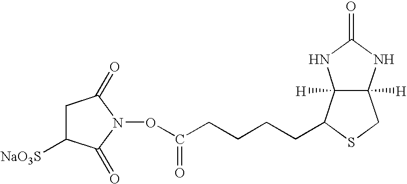 |
| 3 | N-hydroxysuccinimide long chain biotin | 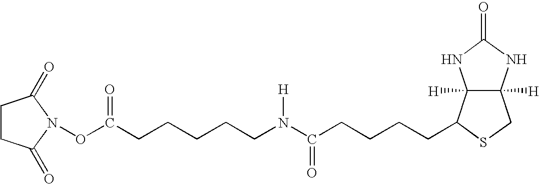 |
| 4 | sulfo-N-hydroxysuccinimide long chain biotin | 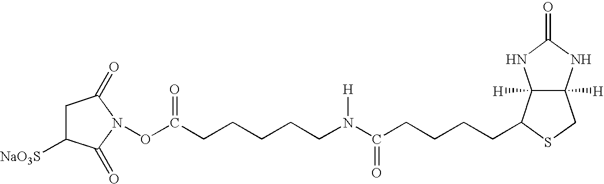 |
| 5 | D-biotin | 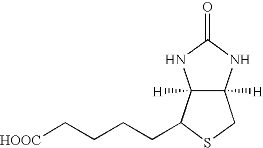 |
| 6 | Biocytin | 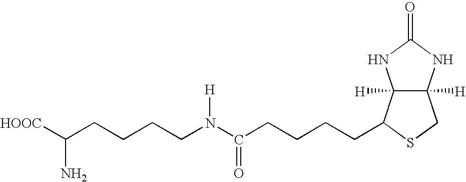 |
| 7 | sulfo-N-hydroxysuccinimide-S- S-biotin | 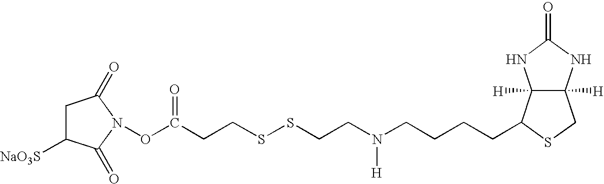 |
| 8 | biotin-BMCC | 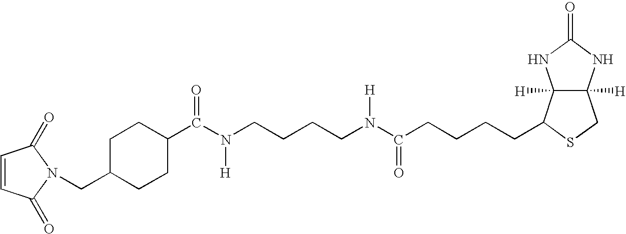 |
| 9 | biotin-HPDP | 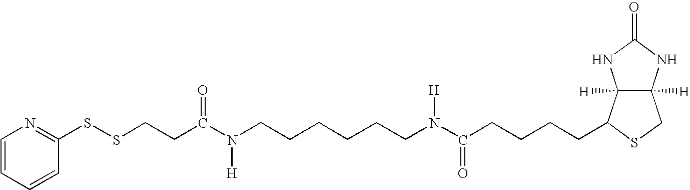 |
| 10 | iodoacetyl-LC-biotin | 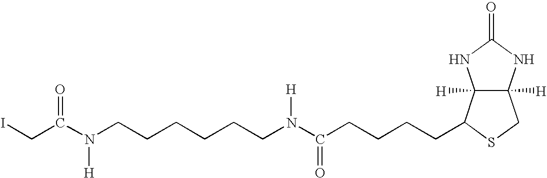 |
| 11 | biotin-hydrazide | 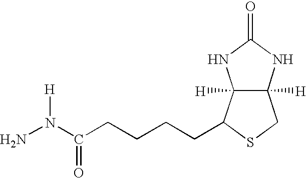 |
| 12 | biotin-LC-hydrazide | 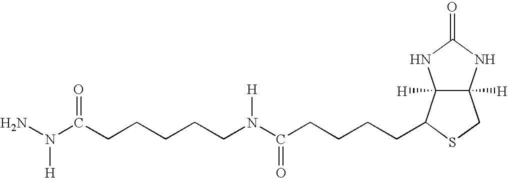 |
| 13 | biocytin hydrazide | 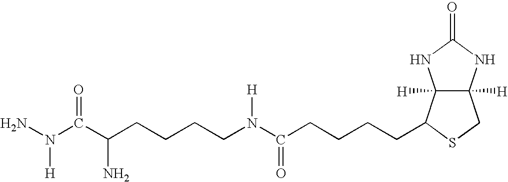 |
| 14 | biotin cadaverine | 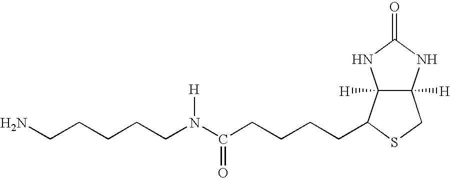 |
| 15 | Carboxybiotin | 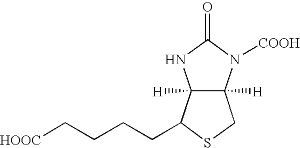 |
| 16 | photobiotin | 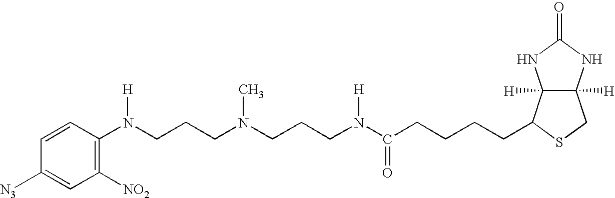 |
| 17 | ρ-aminobenzoyl biocytin trifluoroacetate | 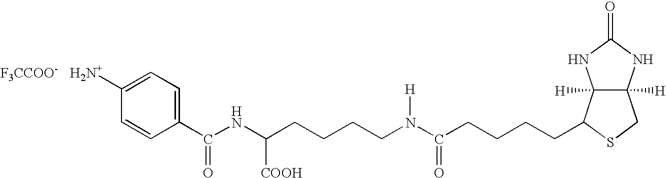 |
| 18 | ρ-diazobenzoyl biocytin | 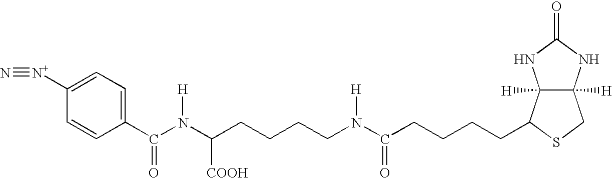 |
| 19 | biotin DHPE | 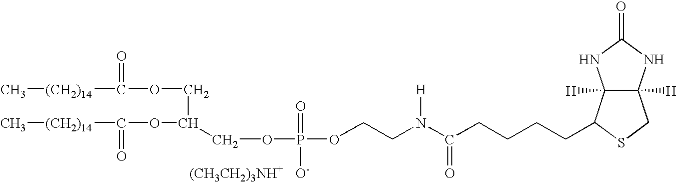 |
| 20 | biotin-X-DHPE | 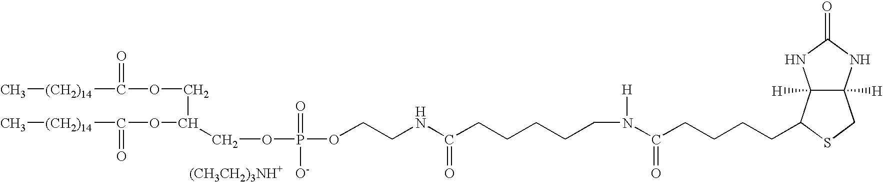 |
| 21 | 12-((biotinyl)amino)dodecanoic acid | 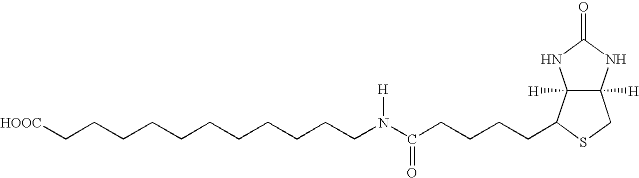 |
| 22 | 12-((biotinyl)amino)dodecanoic acid succinimidyl ester | 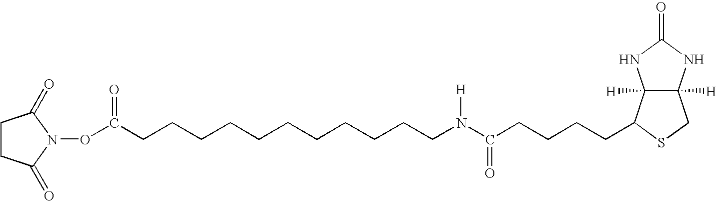 |
| 23 | S-biotinyl homocysteine | 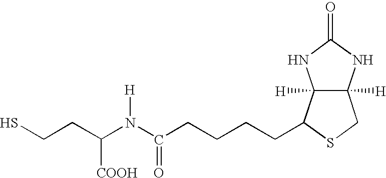 |
| 24 | biocytin-X | 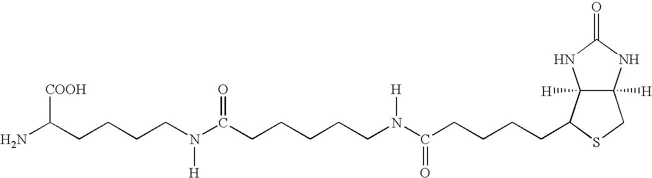 |
| 25 | biocytin x-hydrazide | 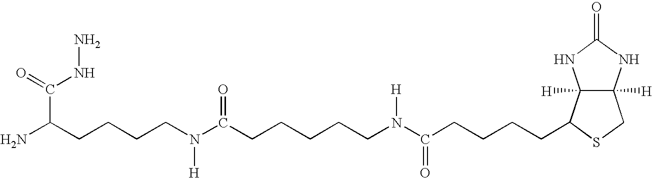 |
| 26 | Biotinethylenediamine | 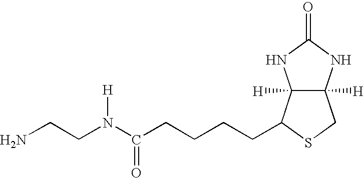 |
| 27 | biotin-X | 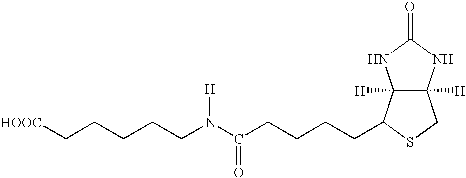 |
| 28 | biotin-X-ethylenediamine | 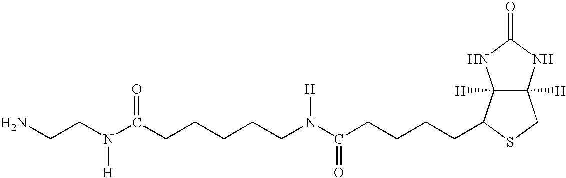 |
| 29 | biotin-XX hydrazide | 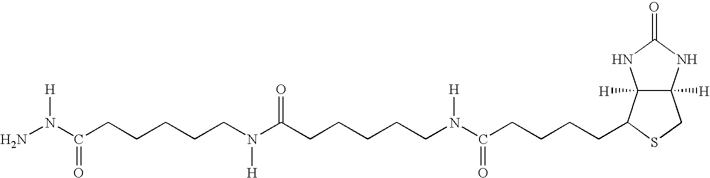 |
| 30 | biotin-XX-SE | 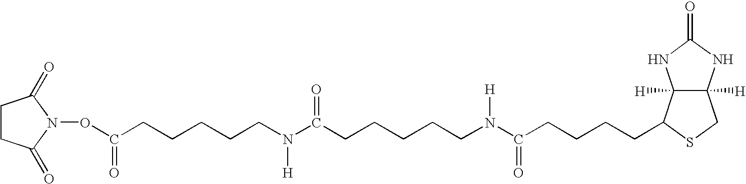 |
| 31 | biotin-XX,SSE | 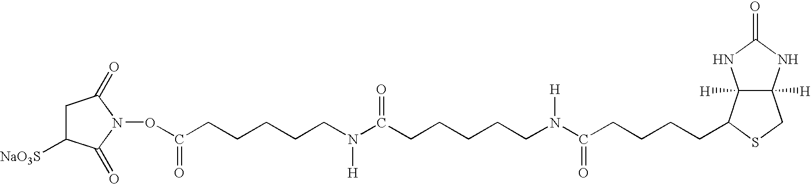 |
| 32 | biotin-X-cadaverine | 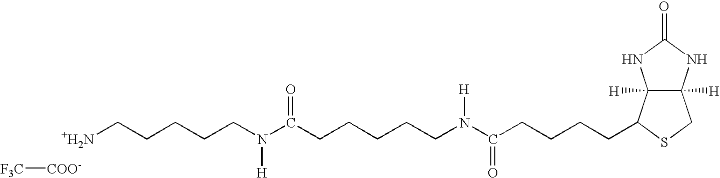 |
| 33 | α-(t-BOC)biocytin | 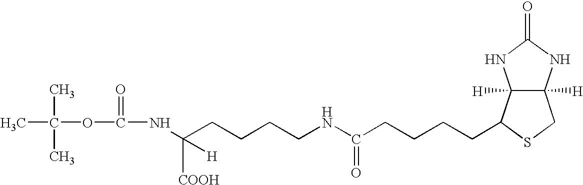 |
| 34 | N-(biotinyl)-N′- (iodoacetyl)ethylenediamine | 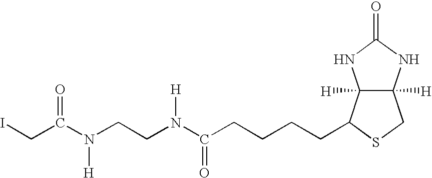 |
| 35 | DNP-X-biocytin-X-SE | 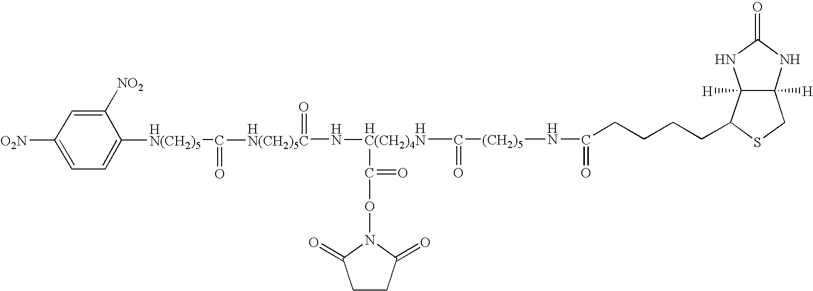 |
| 36 | biotin-X-hydrazide | 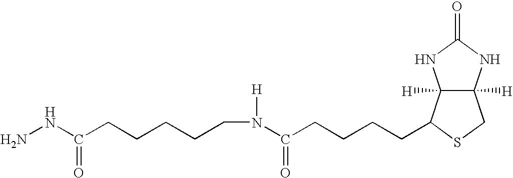 |
| 37 | norbiotinamine hydrochloride | 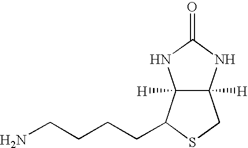 |
| 38 | 3-(N-maleimidylpropionyl) biocytin | 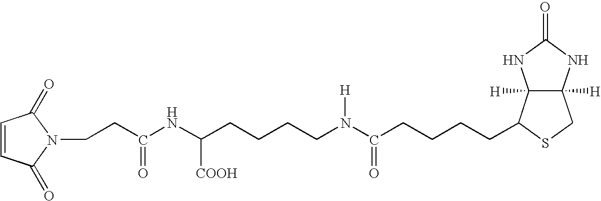 |
| 39 | ARP; N-(aminooxyacetyl)-N′-(D- biotinyl)hydrazine | 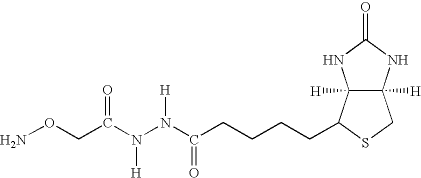 |
| 40 | biotin-1-sulfoxide | 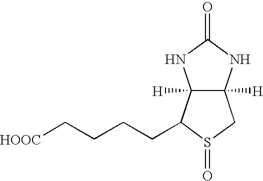 |
| 41 | biotin methyl ester | 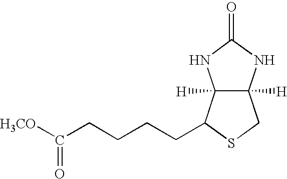 |
| 42 | biotin-maleimide | 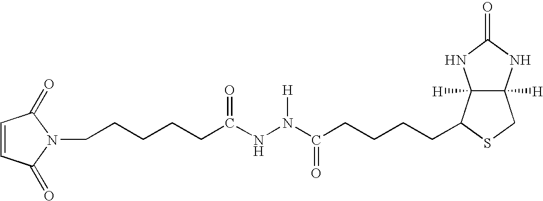 |
| 43 | Biotin- poly(ethyleneglycol)amine | 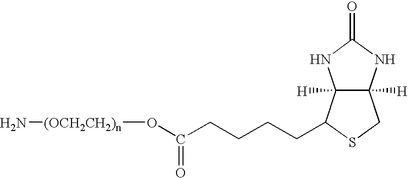 |
| 44 | (+) biotin 4-amidobenzoic acid sodium salt | 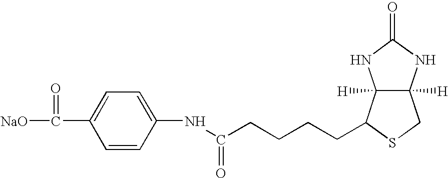 |
| 45 | Biotin 2-N-acetylamino-2- deoxy-β-D-glucopyranoside | 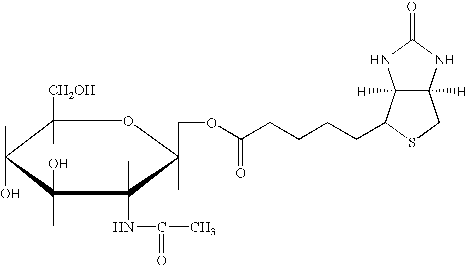 |
| 46 | Biotin-α-D-N-acetylneuraminide | 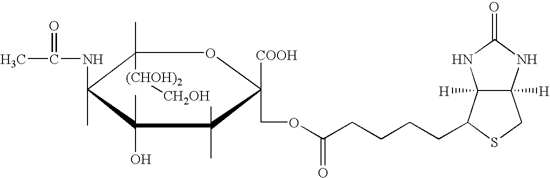 |
| 47 | Biotin-α-L-fucoside | 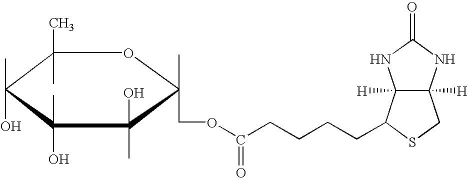 |
| 48 | Biotin lacto-N-bioside | 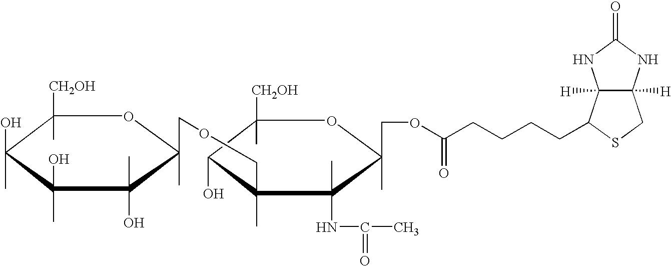 |
| 49 | Biotin-Lewis-A trisaccharide | 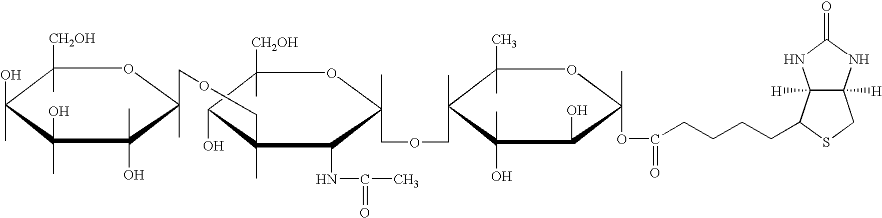 |
| 50 | Biotin-Lewis-Y tetrasaccharide | 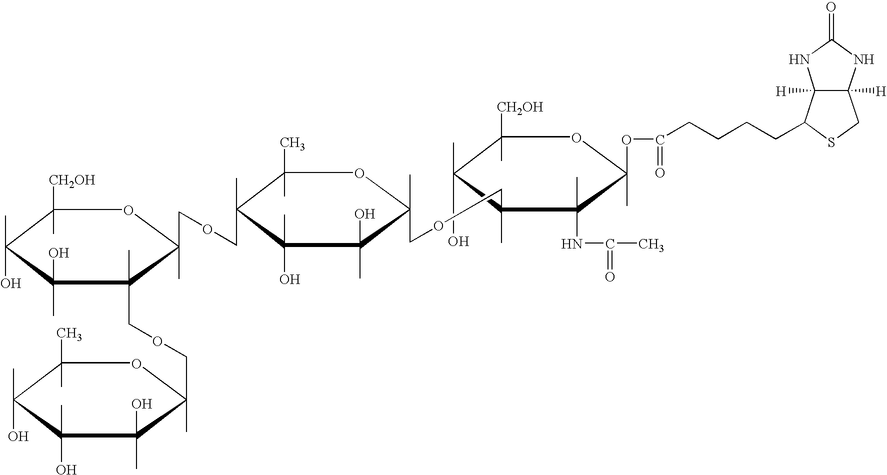 |
| 51 | Biotin-α-D-mannopyranoside | 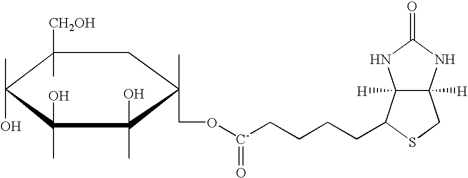 |
| 52 | biotin 6-O-phospho-α-D- mannopyranoside | 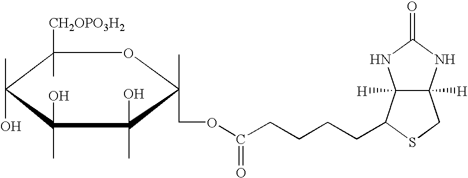 |
| 53 | polychromium-poly(bis)-[N- (2,6-(diisopropylphenyl) carbamoyl methyl)imino diacetic acid] | 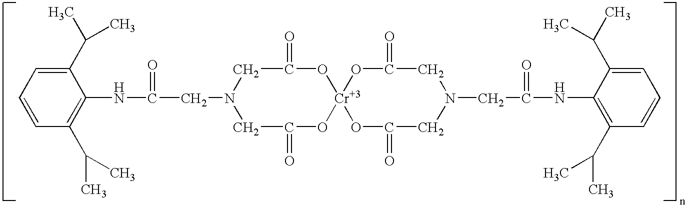 |

Structure of iminobiotin compounds are not shown in Table 3. The iminobiotin structures are analogs of the biotin structure where the biotin group is replaced by a an iminobiotin group. An example is shown below with the analogs N-hydroxysuccinimide biotin and N-hydroxysuccinimide iminobiotin. 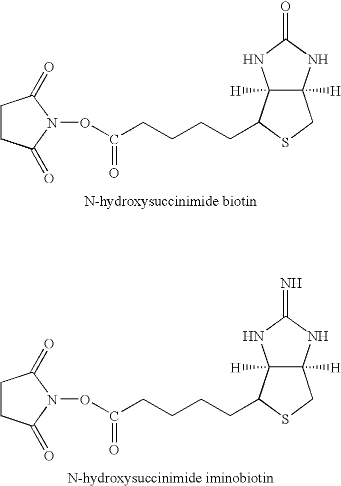
carbonyl carbon and a hydroxyl functionality, participates in hydrogen bonding with neighboring negative and positive charged dipoles residing on interferon and various lipid molecules.

In an embodiment, cellulose acetate hydrogen phthalate polymer interacts with the lipids through ion-dipole bonding with 1,2-distearoyl-sn-glycero-3-phosphocholine phosphate and dicetyl phosphate molecules. The ion-dipole bonding occurs between the δ⁺ hydrogen on the hydroxyl groups of cellulose and the negatively charged oxygen atom on the phosphate moiety of the phospholipid molecules. The functional groups with the largest role in the ion-dipole interaction are the negatively charged oxygen atoms on the phosphate groups of the phospholipid molecules, hydrogen atoms on the hydroxyl groups and the hydrogen atoms on amide bonds of the interferon molecules. Negatively charged functional groups form sites for ion-dipole interactions and for reacting with the δ⁺ hydrogen atom on individual hydroxyl groups and the hydroxyl groups of the carboxyl functionalities on cellulose acetate hydrogen phthalate. Ion-dipoles can be formed between the positively charged quaternary amines on the phosphocholine functionalities and the δ⁻ carbonyl oxygen found on cellulose acetate hydrogen phthalate and interferon. Sugar molecules comprising branched hydrophilic structures in interferon can participate in hydrogen bonding and ion-dipole interactions.

The molecular configuration and the size of the polymer (with an approximate molecular weight of 15,000 or more) can enable cellulose acetate hydrogen phthalate to coat individual phospholipid molecules of the supra-molecular lipid construct in the region of the hydrophilic head group. This coating can protect interferon within the supra-molecule lipid construct from the acid milieu of the stomach. This is the first time that individual proteinaceous and lipid molecules have been protected from hydrolytic degradation in the stomach milieu. There are several ways that cellulose acetate hydrogen phthalate can be attached to the surface of molecules within the supra-molecule lipid construct. A preferred means of linking cellulose acetate hydrogen phthalate to the surface of the lipid construct is to attach the polymeric cellulosic species to a tail of an interferon molecule that presents a sugar that projects from the surface of the supra-molecule lipid construct. This will protect the interferon proteinaceous tails from enzymatic hydrolysis.

An extended amphipathic lipid comprises a variety of multi-dentate binding sites for attachment to the receptor. Multi-dentate binding, as defined herein, requires a plurality of potential binding sites on the surface of interferon and its accompanying sugar moieties, as well as on the supra-molecular lipid construct that can interface with carbonyl, carboxyl and hydroxyl functional groups on the cellulose acetate hydrogen phthalate polymer. This enables the cellulose acetate hydrogen phthalate polymer to bind to a plurality of hydrophilic regions not only on the supra-molecular lipid construct but also on molecules of interferon in order to establish a shield of hydrolytic protection for the lipid construct. In this manner both interferon and the supra-molecule lipid construct are protected from the acid environment of the stomach following oral administration of the interferon dosage form. Even though cellulose acetate hydrogen phthalate covers or shields individual lipid molecules within and on the surface of the supra-molecule lipid construct while passing through the stomach, once the construct migrates to the alkaline region of the small intestine, cellulose acetate hydrogen phthalate is hydrolytically degraded. After cellulose acetate hydrogen phthalate is removed from the surface of the molecules of the supra-molecule lipid construct, a lipid anchoring-hepatocyte receptor binding molecule, such as 1,2-dipalmitoyl-sn-glycero-3-phosphoethanolamine-N-(Cap Biotinyl), becomes exposed and then is available to bind with the receptor. The employment of a cellulose acetate hydrogen phthalate coating on interferon and the supra-molecular lipid construct is needed to ensure that a greater bioavailability of interferon is achieved.

In an embodiment, the supra-molecule lipid construct comprises a target molecule complex comprising multiple linked individual units formed by complexing a bridging component with a complexing agent. The bridging component is a water soluble salt of a metal capable of forming a water-insoluble coordinated complex with a complexing agent. A suitable metal is selected from the transition and inner transition metals or neighbors of the transition metals. The transition and inner transition metals from which the metal are selected from: Sc (scandium), Y (yttrium), La (lanthanum), Ac (actinium), the actinide series; Ti (titanium), Zr (zirconium), Hf (hafnium), V (vanadium), Nb (niobium), Ta (tantalum), Cr (chromium), Mo (molybdenum), W (tungsten), Mn (manganese), Tc(technetium), Re (rhenium), Fe (iron), Co (cobalt), Ni (nickel), Ru (ruthenium), Rh (rhodium), Pd (palladium), Os (osmium), Ir (iridium), and Pt (platinum). The neighbors of the transition metals from which the metal can be selected are: Cu (copper), Ag (silver), Au (gold), Zn (zinc), Cd (cadmium), Hg (mercury), Al (aluminum), Ga (gallium), In (indium), TI (thallium), Ge (germanium), Sn (tin), Pb (lead), Sb (antimony) and Bi (bismuth), and Po (polonium). Examples of metal compounds useful as bridging agents include chromium chloride (III) hexahydrate; chromium (III) fluoride tetrahydrate; chromium (III) bromide hexahydrate; zirconium (IV) citrate ammonium complex; zirconium (IV) chloride; zirconium (IV) fluoride hydrate; zirconium (IV) iodide; molybdenum (III) bromide; molybdenum (III) chloride; molybdenum (IV) sulfide; iron (III) hydrate; iron (III) phosphate tetrahydrate, iron (III) sulfate pentahydrate, and the like.

The complexing agent is a compound capable of forming a water insoluble coordinated complex with a bridging component. There are several families of suitable complexing agents.

A complexing agent can be selected from the family of iminodiacetic acids of the formula (1) where R₁ is loweralkyl, aryl, arylloweralkyl, and a heterocyclic substituent.

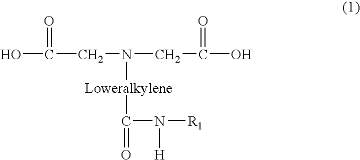

Suitable compounds of the formula (1) include:
N-(2,6-diisopropylphenylcarbamoylmethyl)iminodiacetic acid;
N-(2,6-diethylphenylcarbamoylmethyl)iminodiacetic acid;
N-(2,6-dimethylphenylcarbamoylmethyl)iminodiacetic acid;
N-(4-isopropylphenylcarbamoylmethyl)iminodiacetic acid;
N-(4-butylphenylcarbamoylmethyl)iminodiacetic acid;
N-(2,3-dimethylphenylcarbamoylmethyl)iminodiacetic acid;
N-(2,4-dimethylphenylcarbamoylmethyl)iminodiacetic acid;
N-(2,5-dimethylphenylcarbamoylmethyl)iminodiacetic acid;
N-(3,4-dimethylphenylcarbamoylmethyl)iminodiacetic acid;
N-(3,5-dimethylphenylcarbamoylmethyl)iminodiacetic acid;
N-(3-butylphenylcarbamoylmethyl)iminodiacetic acid;
N-(2-butylphenylcarbamoylmethyl)iminodiacetic acid;
N-(4-tertiary butylphenylcarbamoylmethyl)iminodiacetic acid;
N-(3-butoxyphenylcarbamoylmethyl)iminodiacetic acid;
N-(2-hexyloxyphenylcarbamoylmethyl)iminodiacetic acid;
N-(4-hexyloxyphenylcarbamoylmethyl)iminodiacetic acid; aminopyrrol iminodiacetic acid;
N-(3-bromo-2,4,6-trimethylphenylcarbamoylmethyl)iminodiacetic acid; benzimidazole methyl iminodiacetic acid;
N-(3-cyano-4,5-dimethyl-2-pyrrylcarbamoylmethyl)iminodiacetic acid;
N-(3-cyano-4-methyl-5-benzyl-2-pyrrylcarbamoylmethyl)iminodiacetic acid; and
N-(3-cyano-4-methyl-2-pyrrylcarbamoylmethyl)iminodiacetic acid and other derivatives of N-(3-cyano-4-methyl-2-pyrrylcarbamoylmethyl)iminodiacetic acid of formula (2),

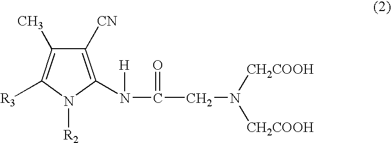

where R₂ and R₃ are the following:

| R₂ | R₃ |
|---|---|
| H | iso-C₄H₉ |
| H | CH₂CH₂SCH₃ |
| H | CH₂C₆H₄-p-OH |
| CH₃ | CH₃ |
| CH₃ | iso-C₄H₉ |
| CH₃ | CH₂CH₂SCH₃ |
| CH₃ | C₆H₅ |
| CH₃ | CH₂C₆H₅ |
| CH₃ | CH₂C₆H₄-p-OCH₃ |

A complexing agent can be selected from the family of imino diacid derivatives of the general formula (3), where R₄, R₅, and R₆ are independent of each other and can be hydrogen, loweralkyl, aryl, arylloweralkyl, alkoxyloweralkyl, and heterocyclic.

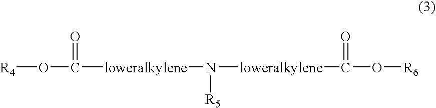

Suitable compounds of the formula (3) include: N′-(2-acetylnaphthyl) iminodiacetic acid (NAIDA); N′-(2-naphthylmethyl) iminodiacetic acid (NMIDA); iminodicarboxymethyl-2-naphthylketone phthalein complexone; 3 (3: 7a: 12a: trihydroxy-24-norchol anyl-23-iminodiacetic acid; benzimidazole methyl iminodiacetic acid; and N-(5,pregnene-3-p-ol-2-oyl carbamoylmethyl) iminodiacetic acid.

A complexing agent can be selected from the family of amino acids of formula (4),

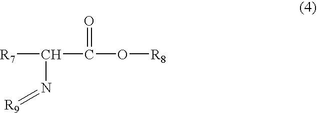

where R₇ is an amino acid side chain, R₈ is loweralkyl, aryl, arylloweralkyl, and R₉ is pyridoxylidene.

Suitable amino acids of the formula (4) are aliphatic amino acids, including, but not limited to: glycine, alanine, valine, leucine, isoleucine; hydroxyamino acids, including serine, and threonine; dicarboxylic amino acids and their amides, including aspartic acid, asparagine, glutamic acid, glutamine; amino acids having basic functions, including lysine, hydroxylysine, histidine, arginine; aromatic amino acids, including phenylalanine, tyrosine, tryptophan, thyroxine; and sulfur-containing amino acids, including cystine, methionine.

A complexing agents can be selected from amino acid derivatives including, but are not necessarily limited to (3-alanine-y-amino) butyric acid, O-diazoacetylserine (azaserine), homoserine, omithine, citrulline, penicillamine and members of the pyridoxylidene class of compounds including, but are not limited to: pyridoxylidene glutamate; pyridoxylidene isoleucine; pyridoxylidene phenylalanine; pyridoxylidene tryptophan; pyridoxylidene-5-methyl tryptophan; pyridoxylidene-5-hydroxytryptamine; and pyridoxylidene-5-butyltryptamine.

A complexing agent can be selected from the family of diamines of the general formula (6), 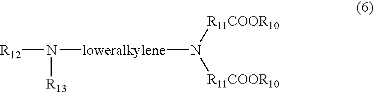
where R₁₀ is hydrogen, loweralkyl, or aryl; R₁₁ is loweralkylene or arylloweralky; R₁₂ and R₁₃ independently are hydrogen, loweralkyl, alkyl, aryl, arylloweralkyl, acylheterocyclic, toluene, sulfonyl or tosylate.

Some suitable diamines of the formula (6) include, but are not limited to, ethylenediamine-N, N diacetic acid; ethylenediamine-N,N-bis (-2-hydroxy-5-bromophenyl) acetate; N′-acetylethylenediamine-N,N diacetic acid; N′-benzoyl ethylenediamine-N,N diacetic acid; N′-(p-toluenesulfonyl) ethylenediamine-N, N diacetic acid; N′-(p-t-butylbenzoyl) ethylenediamine-N, N diacetic acid; N′-(benzenesulfonyl) ethylenediamine-N, N diacetic acid; N′-(p-chlorobenzenesulfonyl) ethylenediamine-N, N diacetic acid; N′-(p-ethylbenzenesulfonyl ethylenediamine-N, N diacetic acid; N′-acyl and N′sulfonyl ethylenediamine-N, N diacetic acid; N′-(p-n-propylbenzenesulfonyl) ethylenediamine-N, N diacetic acid; N′-(naphthalene-2-sulfonyl) ethylenediamine-N, N diacetic acid; and N′-(2, 5-dimethylbenzenesulfonyl) ethylenediamine-N, N diacetic acid.

Other suitable complexing compounds or agents include, but are not limited to: penicillamine; p-mercaptoisobutyric acid; dihydrothioctic acid; 6-mercaptopurine; kethoxal-bis(thiosemicarbazone); Hepatobiliary Amine Complexes, 1-hydrazinophthalazine (hydralazine); sulfonyl urea; Hepatobiliary Amino Acid Schiff Base Complexes; pyridoxylidene glutamate; pyridoxylidene isoleucine; pyridoxylidene phenylalanine; pyridoxylidene tryptophan; pyridoxylidene 5-methyl tryptophan; pyridoxylidene-5-hydroxytryptamine; pyridoxylidene-5-butyltryptamine; tetracycline; 7-carboxy-p-hydroxyquinoline; phenolphthalein; eosin I bluish; eosin I yellowish; verograffin; 3-hydroxyl-4-formyl-pyridene glutamic acid; Azo substituted iminodiacetic acid; hepatobiliary dye complexes, such as rose bengal; congo red; bromosulfophthalein; bromophenol blue; toluidine blue; and indocyanine green; hepatobiliary contrast agents, such as iodipamide; and ioglycamic acid; bile salts, such as bilirubin; cholgycyliodohistamine; and thyroxine; hepatobiliary thio complexes, such as penicillamine; p-mercaptoisobutyric acid; dihydrothiocytic acid; 6-mercaptopurine; and kethoxal-bis (thiosemicarbazone); hepatobiliary amine complexes, such as 1-hydrazinophthalazine (hydralazine); and sulfonyl urea; hepatobiliary amino acid Schiff Base complexes, including pyridoxylidene-5-hydroxytryptamine; and pyridoxylidene-5-butyltryptamine; hepatobiliary protein complexes, such as protamine; ferritin; and asialo-orosomucoid; and asialo complexes, such as lactosaminated albumin; immunoglobulins, G, IgG; and hemoglobin.

The three-dimensional target molecule complex made from combining bridging agents and complexing agents is described in WO 99/59545, which is incorporated by reference. In an embodiment, the bridging agent is a metal salt, such as chromium chloride hexahydrate, capable of forming a coordinated complex with complexing agents, such as N-(2,6-diisopropylphenylcarbamoylmethyl) iminodiacetic acid. The bridging agent and the complexing agents are combined to form a complex composed of multiple linked units in a three-dimensional array. In a preferred embodiment, the complex is composed of multiple units of chromium (bis) [N-(2,6-(diisopropylphenyl)carbamoyl methyl)imino diacetic acid] linked together. In an embodiment, the chromium target molecule complex substance is soluble in a mixture of lipids containing 1,2-distearoyl-sn-glycero-3-phosphocholine, dicetyl phosphate and cholesterol. The complex is incorporated within a supra-molecular lipid construct formed from the groups of lipids previously described.

In an embodiment, interferon can be mixed in an appropriate proportion with antiviral agents, such as ribivirin, acyclovir, double stranded DNA, oligonucleotides, protease inhibitors, reverse transcriptase inhibitors and other possible anti-viral materials that are ineffective by themselves, but effective when delivered in an HDV.

Description of the Invention—Method of Manufacturing the Supra-Molecular Construct

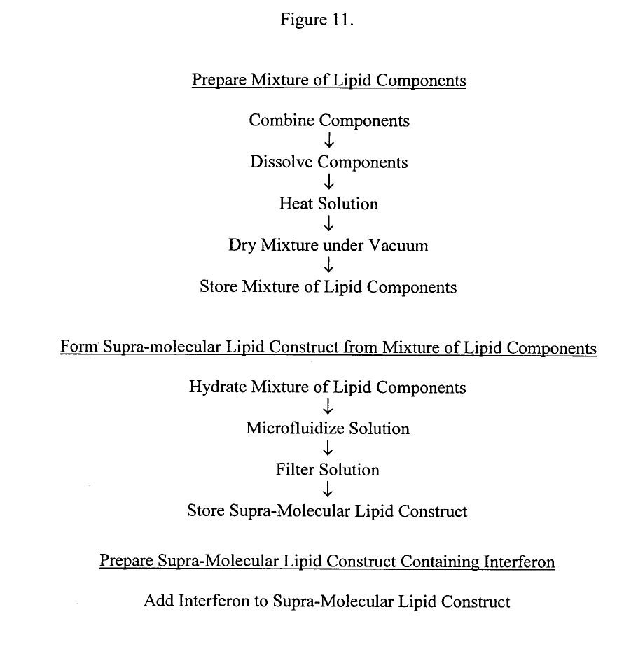

FIG. 11 demonstrates an outline for the process for manufacturing a supra-molecular construct comprising amphipathic lipid molecules, an extended amphipathic lipid and interferon.

The manufacture of the composition comprises three overall steps: preparing a mixture of amphipathic lipid molecules and an extended amphipathic lipid, preparing a supra-molecular construct from the mixture of amphipathic lipid molecules and an extended amphipathic lipid, and combining interferon into the supra-molecular construct.

Lipids can be produced and loaded by the methods disclosed herein, and those methods described in U.S. Pat. Nos. 4,946,787; 4,603,044; and 5,104,661, and the references cited therein. Typically, the aqueous supra-molecular lipid construct formulations of this invention will comprise 0.1% to 10% active agent by weight (i.e. 1-10 mg drug per ml), and 0.1% to 4% lipid by weight in an aqueous solution, optionally containing salts and buffers, in a quantity to make 100% by volume. Preferred are formulations which comprise 0.1% to 5% active agent. Most preferred is a formulation comprising 0.01% to 5% active agent by weight and up to 2% by weight of a lipid component in an amount of aqueous solution sufficient (q. s.) to make 100% by volume.

In an embodiment, the supra-molecular lipid construct can be prepared by the following procedure. Individual lipid constituents are mixed together in an organic solvent system where the solvent had been dried over molecular sieves for approximately two hours to remove any residual water that may have accompanied the solvent. In an embodiment, the solvent system comprises a mixture chloroform and methanol in the ratio 2:1 by volume. Other organic solvents that can be easily removed from a mixture of dried lipids can be used. Use of a single-step addition of the lipid constituents in the initial mixing procedure obviates the need for introducing any additional coupling reactions which would unnecessarily complicate the structure of the supra-molecular lipid construct and require additional separation procedures. The lipid components and the hepatocyte receptor binding molecule are dissolved in the solvent, then the solvent is removed under high vacuum until a dried mixture of the lipids forms. In an embodiment, the solvent is removed under vacuum using a rotoevaporator, or other methods known in the art, with slow turning at approximately 60° C. for approximately two hours. This mixture of lipids can be stored for further use, or directly used.

The supra-molecular construct is prepared from the dried mixture of amphipathic lipid molecules and an extended amphipathic lipid. The dried mixture of lipids are added to an appropriate amount of aqueous buffered media, then the mixture is swirled to form a homogeneous suspension. The lipid mixture is then heated with mixing at approximately 80° C. for approximately 30 minutes under a dry nitrogen atmosphere. The heated homogeneous suspension is immediately transferred to a micro-fluidizer preheated to approximately 70° C. The suspension is passed through the microfluidizer. The suspension may require additional passes through the microfluidizer to obtain a homogeneous lipid micro-suspension. In an embodiment a Model #M-110 EHI micro-fluidizer was used where the pressure on the first pass was approximately 9,000 psig. A second pass of the lipid suspension through the micro-fluidizer may be needed to produce a product that exhibits the properties of a homogeneous lipid micro-suspension. This product is defined structurally and morphologically as a three-dimensional supra-molecular lipid construct which contains a hepatocyte receptor binding molecule.

Interferon can be loaded into the supra-molecular lipid constructs by two methods: equilibrium loading and non-equilibrium loading. Equilibrium loading of interferon begins when interferon is added to a suspension of the supra-molecular lipid constructs. Over time, interferon molecules move into and out of the supra-molecular lipid construct. The movement is governed by partitioning equilibrium, where movement into the supra-molecular lipid construct after the initial introduction of interferon to the suspension.

Non-equilibrium loading of interferon into the supra-molecular lipid constructs localizes interferon within the supra-molecular lipid construct. Following equilibrium loading of free interferon into the supra-molecular lipid construct, the bulk phase media that contains free interferon is removed. The non-equilibrium loading procedure is a vector-driven process that begins the instant the external bulk phase media is removed. The gradient potential for interferon to migrate out of the supra-molecular lipid constructs is eliminated when the aqueous phase containing interferon has been removed. The overall process results in a greater concentration of interferon within the final supra-molecular lipid construct because movement of interferon from within the construct is eliminated. The equilibrium loading of interferon is a time-dependent phenomenon whereas the non-equilibrium loading procedure is practically instantaneous. Non-equilibrium loading can be initiated by a variety of processes where the material in solution is separated from the supra-molecular lipid construct. Examples of such processes include, but are not limited to: filtration, centricon centrifugation, batch style affinity chromatography, streptavidin agarose affinity-gel chromatography or batch style ion-exchange chromatography. Any means that eliminates the gradient potential for interferon diffusion and leakage and causes the interferon to be retained by the supra-molecular construct can be utilized.

When using batch-style chromatography, the affinity or ion-exchange gel is mixed rapidly with the mixture of interferon and the construct. Binding to the chromatography medium occurs rapidly and the chromatography medium can be removed from the aqueous media by decanting of the aqueous phase or by using classic filtering techniques such as the use of filter paper and a Büchner funnel.

The supra-molecular lipid construct contains a discrete amount of loaded interferon located not only inside, but also within and on the surface of the lipid construct. The supra-molecular lipid construct created is a new and novel composition of matter and becomes a composition for delivering an effective amount of interferon as a result of non-equilibrium loading. The loading of interferon into this supra-molecular lipid construct and the subsequent removal of bulk phase interferon results in a high concentration of interferon in a supra-molecular lipid construct by shortening the length of time needed for removal of the external phase media. It would be difficult to achieve this level of loading interferon into the construct using time-dependent procedures, such as ion-exchange or gel-filtration chromatography, since these procedures require a constant infusion of buffer comprising high concentrations of interferon. For example, loading interferon into the construct using small scale column chromatography requires approximately twenty minutes to remove the external bulk phase media containing interferon from the construct containing interferon. Equilibrium conditions are reestablished during this time period by movement of interferon from the construct. Maintaining a high concentration of interferon in and on the supra-molecular lipid construct is one of the positive benefits of using non-equilibrium loading.

In an extension of the non-equilibrium loading process, cellulose acetate hydrogen phthalate can be added to the supra-molecular lipid construct during the step of loading interferon to the supra-molecular lipid construct after the interferon has undergone equilibrium loading but before the non-equilibrium loading process is initiated. The nature and structure of the interferon molecule allows it to be intercalated into the supra-molecule lipid construct were interferon is dispersed throughout the supra-molecule lipid construct. Hydrophilic portions of interferon, as well as branched complex sugars and additional functional groups, extend into the bulk phase media from the surface of the supra-molecular lipid construct. These extended hydrophilic portions of interferon can participate in hydrogen bonding, dipole-dipole and ion-dipole interactions at the surface of the supra-molecule lipid construct with the hydroxyl groups, carboxyl groups and carbonyl functionalities of cellulose acetate hydrogen phthalate as illustrated in FIG. 10. Cellulose acetate hydrogen phthalate offers a unique means of combining with the molecules of the supra-molecule lipid construct to provide an excellent shield for masking the contents of the lipid construct from the digestive milieu of the stomach. The digestive processes in the stomach result from the hydrolytic cleavage of proteinaceous substrates by the enzyme pepsin as well as cleavage by acid hydrolysis. The acidic environment of the stomach degrades free interferon and can hydrolyze the ester bonds that hold the acyl hydrocarbon chains to the glycerol backbone in the phospholipid molecules. Hydrolytic cleavage can also occur on either side of the phosphate functionality in the phosphocholine group. The digestive system changes from the acid region of the stomach to an alkaline region of the small intestine were enzymatic action of trypsin and chymotrypsin occurs. Amino acid lysing enzymes, such as alpha amino peptidases, can degrade proteins such as interferon from the N-terminal end. The presence of cellulose acetate hydrogen phthalate in the supra-molecule lipid construct protects interferon from hydrolytic degradation. As the alkaline environment of the small intestine hydrolytically degrades the cellulose acetate hydrogen phthalate shield of the supra-molecular lipid construct the hepatocyte receptor binding molecule becomes available to direct binding of the construct to the hepatocyte binding receptor. While not wishing to be bound by any particular theory, there is a synergy of hydrolytic protection upon the addition of cellulose acetate hydrogen phthalate at the end point of non-equilibrium loading. The protection is distributed not only to interferon and individual lipid molecules, but also to the entire supra-molecular lipid construct. This synergy provides collective as well as individual molecular protection from enzymatic and acid hydrolysis.

In an embodiment, cellulose acetate hydrogen phthalate can be covalently bound to either interferon or the supra-molecular lipid construct by a variety of methods. For example, a method involves coupling the hydroxyl groups on cellulose acetate hydrogen phthalate with the amine functionalities on either 1,2-diacyl-sn-glycero-3-phosphoethanolamine or the ε-amino group of the ten L-lysines in the interferon molecule utilizing the Mannich reaction.

In an embodiment, cellulose acetate hydrogen phthalate is loaded into the supra-molecular lipid construct during equilibrium loading of interferon into the construct. The hydroxyl and carbonyl functionalities of the cellulose acetate hydrogen phthalate can hydrogen bond with lipid molecules in a supra-molecular lipid construct. Hydrogen bonds between cellulose acetate hydrogen phthalate and the construct are formed concurrently as interferon is loaded under equilibrium conditions into the lipid construct creating a shield around interferon and around the construct.

Interferon can be recovered and recycled from aqueous media by binding it to streptavidin-agarose iminobiotin. Streptavidin covalently bound to cyanogen bromide activated agarose provides a means to separate an iminobiotin-based supra-molecular lipid construct from interferon in the aqueous media at the end of non-equilibrium loading of interferon into the construct. In an embodiment, an iminobiotin derivative forms the hepatocyte receptor binding portion of the phospholipid moiety within the supra-molecular lipid construct. The water-soluble portion of the lipid anchoring molecule extends approximately 30 angstroms from the lipid surface to facilitate binding of the hepatocyte receptor binding portion of the phospholipid moiety with a hepatocyte receptor and to aid in the attachment of the supra-molecular lipid construct to streptavidin.

Streptavidin reversibly binds to iminobiotin at pH values of 9.5 and greater, where the uncharged guandino functional group of iminobiotin strongly binds to one of the four binding sites on streptavidin located approximately nine angstroms below the surface of the protein. A supra-molecular lipid construct containing iminobiotin can be removed from buffered media by raising the pH of an aqueous mixture of the construct to pH 9.5 by the addition of a 20 mM sodium carbonate-sodium bicarbonate buffer. At this pH, the bulk phase media contains free interferon which can be reclaimed and separated from the supra-molecular lipid construct by using a variety of procedures including to, but not limited to: filtration, centrifugation or chromatography.

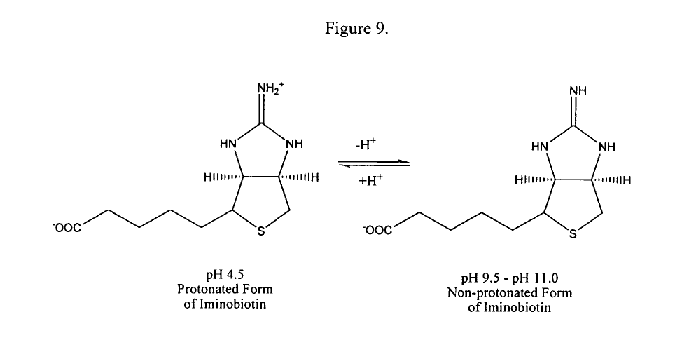

The mixture at pH 9.5 is then mixed with streptavidin-agarose cross-linked beads, where the construct is adsorbed onto the streptavidin. The beads, which are approximately 120 microns in diameter, can be separated from the solution by filtration. The supra-molecular lipid construct is released from the streptavidin-agarose affinity-gel by reducing the pH from pH 9.5 to pH 4.5 by the addition of a 20 mM sodium acetate-acetic acid buffer at pH 4.5. At pH 4.5 the guandino group of iminobiotin becomes protonated and positively charged, as shown in FIG. 9. The lipid construct is released and separated from the streptavidin-agarose bead by filtration. The streptavidin-agarose bead can be reclaimed for additional usage. Thus both free interferon and streptavidin-agarose are conserved and can be re-used.

In an embodiment, a composition that provides for the extended release of interferon can be produced when iminobiotin or iminobiocytin lipid constructs are loaded with interferon alpha using streptavidin-agarose beads. When the pH of the forementioned construct is adjusted from pH 9.5 to pH 4.5 interferon-alpha will precipitate within the supra-molecular lipid construct at approximately pH 5.9. The isoelectric point of interferon-alpha is at pH 5.9 and represents the pH at which interferon-alpha has its lowest water-solubility. Over a pH range from pH 5.9 to pH 6.7 interferon-alpha remains essentially insoluble and exhibits properties that are commonly attributed to particulate matter. The insolubilized interferon-alpha within a supra-molecular lipid construct creates a novel interferon-alpha formulation that provides for the time-release of interferon-alpha molecules when administered by subcutaneous injection or through oral dosing. Solubilization of interferon-alpha is initiated as the pH of the lipid construct approaches pH 7.4. The supra-molecular lipid construct could be freeze-dried or kept in a non-aqueous environment prior to dosing. In an aqueous dosage form of interferon-alpha, the pH of the interferon-alpha solution can be maintained at approximately pH 6.5 in order to maintain interferon-alpha in the insoluble form. When interferon-alpha is exposed to an external pH gradient in vivo interferon-alpha can be solubilized and move from the supra-molecular lipid construct, thereby supplying interferon-alpha to other virus-harboring tissues. Interferon remaining with the supra-molecular lipid construct maintains the capability of being directed to the hepatocyte binding receptor on the hepatocytes in the liver. Therefore two forms of interferon-alpha are produced from this particular lipid construct. In an in vivo setting, free and lipid associated interferon-alpha are generated in a time-dependent manner. It is anticipated that the solubilization of interferon-alpha that is lipid associated, as previously described, can be manufactured to release of interferon over a designated time-release period. This could lead to less frequent dosing schedules for patients infected with viruses.

In a preferred embodiment, interferon molecules move into the supra-molecular lipid construct and become sequestered within the lipid domains of the loaded supra-molecular lipid construct. A vector-driven process is employed to move interferon molecules in one direction during the final phase of the interferon loading procedure when the chemical equilibrium is disrupted. During the final phase of interferon loading, the buffer or aqueous media is rapidly removed so that the interferon molecules associated with the supra-molecular lipid construct are deprived of an external media into which to migrate. Removal of the external media effectively quenches the equilibrium between interferon associated with the supra-molecular lipid construct and interferon solubilized in the external media. This process is termed non-equilibrium loading. In an embodiment, a supra-molecular lipid construct can be loaded with interferon using equilibrium methods, an interferon concentration of 273,000 units of interferon per microgram of protein can be selected to initiate the loading procedure. Equilibrium loading continues until the lipid construct is saturated with interferon.

The end process of non-equilibrium loading of interferon into the supra-molecular lipid construct requires using a procedure that separates the solid supra-molecular lipid construct from the buffered media containing free interferon. In an embodiment, a filtration procedure with a very fine micro-pore synthetic membrane is used to separate the lipid construct from the external media. In another embodiment, a centricon centrifugation device equipped with an appropriate filter with a 100,000 molecular weight cut off membrane, such as NanoSep filter can be used to remove the supra-molecular lipid construct from the buffered media containing free interferon. The concentration of interferon in the lipid construct is maintained because bound interferon is no longer in equilibrium with the free interferon molecules located in the bulk phase media that had been removed from the construct. Free interferon which was in solution is available to load other lipid constructs. Thus, the vector-driven process of concentrating interferon within the supra-molecular lipid construct is achieved in one-step in essentially a time-independent procedure.

After the supra-molecular lipid construct is isolated from the bulk phase media, it can range in size from approximately 0.0200 microns to 0.4000 microns in diameter. Supra-molecular lipid constructs comprise different particle sizes that generally follow a Gaussian distribution. The appropriate size of the supra-molecular lipid construct needed to achieve the intended pharmacological efficacy can be selected from lipid constructs that comprise particle sizes in a Gaussian distribution by the hepatocyte binding receptor.

The supra-molecular lipid construct comprising interferon, lipids and the hepatocyte receptor binding molecule is prepared by using a micro-fluidization process that provides a high shear force which degrades larger supra-molecular lipid constructs into smaller constructs. The amphipathic lipid constituents of the supra-molecular lipid construct are 1,2-distearoyl-sn-glycero-3-phosphocholine, cholesterol, dicetyl phosphate, 1,2-dipalmitoyl-sn-glycero-3-phosphoethanolamine-N-(Cap Biotinyl), 1,2-distearoyl-sn-glycero-3-phosphoethanolamine, 1,2-dipalmitoyl-sn-glycero-3-phosphoethanolamine-N-(succinyl), 1,2-dipalmitoyl-sn-glycero-3-[phospho-rac-(1-glycerol)] (sodium salt) and appropriate derivatives thereof whose representative structures are depicted in Table 1.

In an embodiment, a construct comprises a target molecule complex comprising multiple linked individual units formed by complexing a bridging component with a complexing agent. Typically the target molecule complex is formed by combining the selected metal compound, e. g. chromium chloride (III) hexahydrate, with an aqueous buffered solution of the complexing agent. In an embodiment, an aqueous buffered solution of the complexing agent is prepared by dissolving the complexing agent, e.g., N-(2,6-diisopropylphenylcarbamoyl methyl)iminodiacetic acid, in an aqueous buffered solution, e.g., 10 mM sodium acetate buffer at a final pH of 3.2-3.3. The metal compound is added in excess in an amount sufficient to complex with an isolatable portion of the complexing agent, and the reaction is conducted at a temperature of 20° C. to 33° C. for 24 to 96 hours, or until the resultant complex precipitates out of aqueous buffered solution. The precipitated complexing agent, which demonstrates polymeric properties, is then isolated for future use. This complex can be added to the mixture of amphipathic lipid molecules and an extended amphipathic lipid prior to preparing a supra-molecular lipid construct.

Description of the Invention—Method of Use

Patients with hepatitis C are administered an effective amount of a hepatocyte targeted composition comprising a mixture of free interferon and interferon associated with a water insoluble target molecule complex. In an embodiment, interferon can be mixed in an appropriate proportion with antiviral agents, such as ribivirin, acyclovir, double stranded DNA, oligonucleotides, protease inhibitors, reverse transcriptase inhibitors and other possible anti-viral materials that are ineffective by themselves, but effective when delivered in an HDV. In an embodiment, the composition can be administered by a subcutaneous or oral route.

After the composition is administered to a patient by subcutaneous injection, the in situ environment of physiological pH in the injection area produces an increase in the pH that affects the morphology and chemical structures of free interferon and the interferon associated with the water insoluble target molecule complex. As the pH of the environment around interferon increases, interferon changes into a soluble form within and attached to a supra-molecular construct where it can move via the circulatory system to the liver.

Oral administration of a pharmaceutical composition comprising interferon associated with a target molecule complex is followed by intestinal absorption of interferon associated with the target molecule complex into the circulatory system of the body where it is also exposed to the physiological pH of the blood. The supra-molecular lipid construct is targeted for delivery to the liver. In an embodiment, the supra-molecular lipid construct is shielded by the presence of cellulose acetate hydrogen phthalate within the construct. With oral administration, the shielded supra-molecular lipid construct transverses the oral cavity, migrates through the stomach and moves into the small intestine where the alkaline pH of the small intestine degrades the cellulose acetate hydrogen phthalate shield. The de-shielded supra-molecular lipid construct is absorbed into the circulatory system. This enables the supra-molecular lipid construct to be delivered to the sinusoids of the liver. A receptor binding molecule, such as 1,2-dipalmitoyl-sn-glycero-3-phosphoethanolamine-N-(Cap Biotinyl) or other forementioned hepatocyte specific molecules, provides a means for the supra-molecular lipid construct to bind to the receptor and then be engulfed or endocytosed by the hepatocytes. Interferon is then released from the supra-molecular lipid construct where, upon gaining access to the cellular environment, it performs its designated function with regard to acting as an agent to counteract infecting viruses such as hepatitis B, hepatitis C, hepatitis D, hepatitis E, hepatitis F, and hepatitis G and other viruses.

The supra-molecular lipid construct structure of this invention provides a useful agent for pharmaceutical application for administering interferon to a host. Accordingly, the structures of this invention are useful as pharmaceutical compositions in combination with pharmaceutically acceptable carriers. Administration of the structures described herein can be via any of the accepted modes of administration for interferon that are desired to be administered. These methods include oral, parenteral, nasal and other systemic or aerosol forms.

The amount of interferon administered will be dependent on the subject being treated, the type and severity of the affliction, the manner of administration and the judgment of the prescribing physician. Although effective dosage ranges for specific biologically active substances of interest are dependent upon a variety of factors, and are generally known to one of ordinary skill in the art, some dosage guidelines can be generally defined. For most forms of administration, the lipid component will be suspended in an aqueous solution and generally not exceed 4.0% (w/v) of the total formulation. The drug component of the formulation will most likely be less than 20% (w/v) of the formulation and generally greater than 0.01% (w/v).

Dosage forms or compositions containing active ingredient in the range of 0.005% to 5% with the balance made up from non-toxic carriers may be prepared.

The exact composition of these formulations may vary widely depending on the particular properties of the drug in question. However, they will generally comprise from 0.01% to 5%, and preferably from 0.05% to 1% active ingredient for highly potent drugs, and from 2%-4% for moderately active drugs.

The percentage of active compound contained in such parenteral compositions is highly dependent on the specific nature thereof, as well as the activity of the compound and the needs of the subject. However, percentages of active ingredient of 0.01% to 5% in solution are employable, and will be higher if the composition is a solid which will be subsequently diluted to the above percentages. Preferably the composition will comprise 0.2%-2.0% of the active agent in solution.

The formulations of the pharmaceutical compositions described herein may be prepared by any method known or hereafter developed in the art of pharmacology. In general, such preparatory methods include the step of bringing the active ingredient into association with a carrier or one or more other ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, parenteral, pulmonary, intranasal, buccal, or another route of administration.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage. However, delivery of the active agent as set forth in this invention may be as low as 1/10, 1/100 or 1/1,000 or smaller than the dose normally administered because of the targeted nature of the interferon therapeutic agent.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. No. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, kaolin or cellulose acetate hydrogen phthalate.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para- hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a supra-molecular lipid construct preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 microns, and preferably from about 1 to about 6 microns. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 microns and at least 95% of the particles by number have a diameter less than 7 microns. More preferably, at least 95% of the particles by weight have a diameter greater than 1 nanometer and at least 90% of the particles by number have a diameter less than 6 microns. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 microns.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 microns. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 75% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 microns, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1%-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a supra-molecular lipid construct preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remingon's Pharmaceutical Sciences, Mack Publishing Co., Easton, Pa., which is incorporated herein by reference.

Typically dosages of the compound of the invention which may be administered to an animal, preferably a human, range in amount from 1 micrograms to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled physician and will depend upon any number of factors, such as, but not limited to, the type and severity of the disease being treated, the type and age of the animal, etc.

The invention also includes a kit comprising the composition of the invention and an instructional material which describes administering the composition to a tissue of a mammal. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression which can be used to communicate the usefulness of the protein of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the components of the invention or be shipped together with a container which contains the components of the invention. Alternatively, the instructional material may be shipped separately from the container with the intention that the instructional material and the compound be used cooperatively by the recipient.

The pharmaceutical compositions useful for practicing the invention may be administered to deliver a dose equivalent to standard doses of interferon.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, companion animals and other mammals.

Pharmaceutical compositions that are useful in the methods of the invention may be prepared, packaged, or sold in formulations suitable for oral or injectable routes of administration.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Experimental Example 1

### Pharmaceutical Composition 1

The materials and methods used in the experiments presented in this Experimental Example are now described.

A supra-molecular lipid construct comprises a mixture of the lipids 1,2-distearoyl-sn-glycero-3-phosphocholine, cholesterol, dicetyl phosphate, 1,2-distearoyl-sn-glycero-3-phosphoethanolamine, 1,2-dipalmitoyl-sn-glycero-3-phosphoethanolamine-N-(succinyl), 1,2-dipalmitoyl-sn-glycero-3-[phospho-rac-(1-glycerol)] (sodium salt), the receptor binding molecule 1,2-dipalmitoyl-sn-glycero-3-phosphoethanolamine-N-(Cap Biotinyl) and interferon.

### Experimental Example 2

### Pharmaceutical Composition 2

A supra-molecular lipid construct comprises a mixture of the lipids 1,2-distearoyl-sn-glycero-3-phosphocholine, cholesterol, dicetyl phosphate, 1,2-distearoyl-sn-glycero-3-phosphoethanolamine, 1,2-dipalmitoyl-sn-glycero-3-phosphoethanolamine-N-(succinyl), 1,2-dipalmitoyl-sn-glycero-3-[phospho-rac-(1-glycero)] (sodium salt), the receptor binding molecule 1,2-dipalmitoyl-sn-glycero-3-phosphoethanolamine-N-(Cap Biotinyl), interferon-alpha and polychromium-poly(bis)-[N-(2,6-(diisopropylphenyl) carbamoylmethyl)imino] diacetic acid]. The lipid anchoring-hepatocyte receptor binding molecule 1,2-dipalmitoyl-sn-glycero-3-phosphoethanolamine-N-(Cap Biotinyl) and polychromium-poly(bis)-[N-(2,6-(diisopropylphenyl)carbamoyl methyl)imino diacetic acid] had been added to the supra-molecule lipid construct at a level of 1.68%±0.5% by weight and 1.2%±0.5% by weight, respectively.

### Experimental Example 3

### Preparation of a Supra-Molecular Construct Containing Interferon-Alfa

The supra-molecular lipid constructs was formed by preparing a mixture of amphipathic lipid molecules and an extended amphipathic lipid, preparing a supra-molecular lipid construct from the mixture of amphipathic lipid molecules and an extended amphipathic lipid, and combining interferon-alpha into the supra-molecular lipid construct.

A mixture of amphipathic lipid molecules and an extended amphipathic lipid was produced by the following procedure. A mixture of the lipid components [total mass of 8.5316 g] of the supra-molecular lipid construct was prepared by combining aliquots of the lipids 1,2-distearoyl-sn-glycero-3-phosphocholine (5.6881 g), cholesterol crystalline (0.7980 g), dicetyl phosphate (1.5444 g), 1,2-dipalmitoyl-sn-glycero-3-phosphoethanolamine-N-(Cap Biotinyl) (0.1436 g), 1,2-distearoyl-sn-glycero-3-phosphoethanolamine (0.1144 g), 1,2-dipalmitoyl-sn-glycero-3-phosphoethanolamine-N-(succinyl) (0.1245 g) and 1,2-dipalmitoyl-sn-glycero-3-[phospho-rac-(1-glycerol)] (sodium salt)(0.1186 g).

A 100 ml solution of chloroform:methanol (2:1 v:v) was dehydrated over 5.0 grams of molecular sieves. The mixture of the lipid components of supra-molecular lipid construct was placed in a 3 liter flask and 45 mls of the chloroform/methanol solution was added to the lipid mixture. The solution was placed in flask on a rotoevaporator with a water bath at 60° C.±2° C. and turned slowly. The chloroform/methanol solution was removed under vacuum on a rotary evaporator using an aspirator for approximately 45 minutes, followed by a vacuum pump for approximately two hours to remove residual solvent, and the solid mixture of the lipids formed. The dried mixture of lipids can be stored in a freezer at approximately −20° C.-0° C. for an indefinite time period.

The supra-molecular lipid construct was prepared from the mixture of amphipathic lipid molecules and an extended amphipathic lipid using the following procedure. The lipid mixture was mixed with approximately 600 ml of 28.4 mM sodium phosphate (monobasic-dibasic) buffer at pH 7.0. The lipid mixture was swirled, then placed in a heated water bath at 80° C.±4° C. for 30 minutes while slowly turning to hydrate the lipids.

A M-110 EHI microfluidizer was preheated to 70° C.±10° C. using SWI with a pH between 6.5-7.5. The suspension of the hydrated target complex was transferred to the microfluidizer and microfluidized at approximately 9000 psig using one pass of the suspension of the hydrated target molecule complex through the fluidizer. After passing through the microfluidizer, an unfiltered sample (2.0-5.0 ml) of the fluidized suspension was collected for particle size analysis using unimodal distribution data from a Coulter N-4 plus particle size analyzer. Prior to all particle size determinations, the sample was diluted with 0.2 micron filtered SWI that has been pH adjusted to between 6.5-7.5. The particle size was required to range from 0.020-0.40 microns. If the particle size was not within this range, the suspension was passed through the microfluidizer again at approximately 9000 psig, and the particle size was analyzed again until the particle size requirements are reached. The microfluidized target molecule complex was collected in a sterile container.

The microfluidized target molecule complex was maintained at 60° C.±2° C. while filtered twice through a sterile 0.8 micron+0.2 micron gang filter attached to a 5.0 ml syringe. An aliquot of the filtered suspension was analyzed to determine the particle size range of particles in the suspension. The particle size range of the final 0.2 micron filtered sample should be in the range from 0.0200-0.2000 microns as determined from the unimodal distribution printout from the particle size analyzer.

Interferon can be loaded into the construct by reverse loading of the construct using the methods described in U.S. Pat. No. 5,104,661, which is incorporated by reference.

### Experimental Example 4

### Method of Use

The efficacy of HDV-interferon alpha was evaluated in a mouse model having a genetic marker response to the hepatic effect of interferon. C57B16 mice were obtained from Jackson Laboratory and a breeding colony was established at Cleveland MetroHealth Center, Cleveland, Ohio. Mice were obtained from the breeding colony. Two groups of mice, a test group and a control group, were treated. The test group received Interferon+HDV, while the control group received Interferon alone. HDV-Interferon comprised 100 mcg HDV to 10 mcg Interferon alpha. HDV was supplied by Hepasome Pharmaceuticals and Roeferon was the source of the interferon alpha. The HDV and interferon alpha were allowed to equilibrate for 12 hours prior to injection into the mice. Mice from both groups were dosed with 100,000 U/kg body wt. To test the timing of response to IFN, Roferon was subcutaneously injected into the mice. The mice were sacrificed at 6 hours after dosing. The spleen and liver of the sacrificed rats were obtained for analysis.

The interferon-stimulated response of the induction of the double stranded RNA dependent protein kinase (PKR) gene was used as a marker of interferon hepatic tissue delivery. The assay used real time quantitative PCR (polymerase chain reaction) to assay the level of PKR messenger ribonucleic acid (mRNA). Oligonucleotide primers corresponding to intron spanning exonic sequence of the mouse PKR mRNA were designed using Oligo V6 software and the sequence confirmed for being unique by subjecting it to blast search at NCBI against genomic and mRNA mouse sequence. More than 30 primer pairs were designed, but only 2 pairs were selected for the experiments. The conditions for the selected pairs were optimized using sequential temperature and magnesium gradients. RNAs were extracted from liver and spleen of animals then reverse transcribed using our proprietary mix ration of random hexamers and oligo-dT and M-MLV RT. The produced cDNAs were subjected to semi-quantitative PCR, the 6 hour time point was selected for the HDV experiments. Two sets of mice (three each) were injected with either HDV-IFN or IFN only in saline. Mice were sacrificed after 6 hours and RNA extracted from liver and spleen and subjected to RT reaction. Real time quantitative PCR was performed on the produced cDNAs using cybr green technology. Comparison of the level of PKR expression level between liver and spleen in HDV-IFN and IFN treated mice were done.

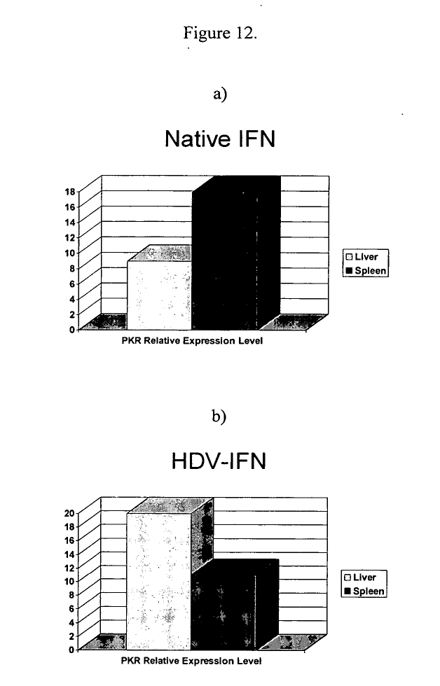

The PKR results are shown in FIG. 12. FIG. 12a indicates the relative expression level in the liver and spleen from mice dosed with interferon alpha. The spleen was selected as a surrogate for evaluating systemic delivery. The relative expression levels in the spleen were compared to the relative expression level in the liver. The relative expression level in the spleen was approximately twice the relative expression level in the liver. FIG. 12b indicates the relative expression level in the liver and spleen from mice dosed with interferon alpha plus HDV. The relative expression level in the liver was approximately twice the relative expression level in the spleen. The relative expression level in the liver of mice treated with HDV-interferon was approximately twice the relative expression level in the liver of mice treated with interferon alone.

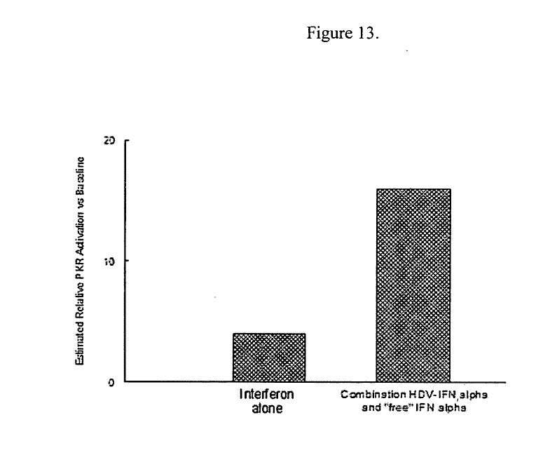

The effect of HDV targeting on hepatic PKR activation by interferon alpha in a mouse model is shown in FIG. 13. Interferon alone provided approximately a 5-fold increase in PKR activation relative to a baseline. HDV-Interferon provided approximately a 15-fold increase in PKR activation relative to a baseline and approximately a 3-fold increase relative to interferon alone. Interferon activity in the hepatic tissue is enhance significantly by delivering the interferon with HDV.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An interferon binding supra-molecular lipid construct comprising amphipathic lipid molecules and an extended amphipathic lipid, wherein said extended amphipathic lipid molecule comprises proximal, medial and distal moieties, wherein said proximal moiety connects said extended lipid molecule to said construct, said distal moiety binds said construct to a hepatocyte binding receptor in the liver, and said medial moiety connects said proximal and distal moieties.

2. The interferon binding supra-molecular lipid construct of claim 1, further comprising at least one interferon selected from the group consisting of interferon-alpha, interferon-alpha-2a, interferon-alpha-2b, interferon-alpha-n1, interferon-alpha-n3, peginterferon alpha 2a, peginterferon alpha 2b, a derivative thereof, or a combination of any of the aforementioned interferons.

3. The interferon binding supra-molecular lipid construct of claim 1, further comprising an insoluble form of interferon associated with the supra-molecular lipid construct.

4. The interferon binding supra-molecular lipid construct of claim 1, further comprising at least one antiviral agent.

5. The interferon binding supra-molecular lipid construct of claim 1, wherein the amphipathic lipid molecules comprise at least one compound selected from the group consisting of 1,2-distearoyl-sn-glycero-3-phosphocholine, cholesterol, dicetyl phosphate, 1,2-dipalmitoyl-sn-glycerol-[3-phospho-rac-(1-glycero)], 1,2-distearoyl-sn-glycero-3-phosphoethanolamine, 1,2-dipalmitoyl-sn-glycero-3-phosphoethanolamine-N-(succinyl), derivatives thereof and mixtures of any of the foregoing compounds.

6. The interferon binding supra-molecular lipid construct of claim 1, further comprising interferon associated with a water insoluble target molecule complex, wherein said complex comprises multiple linked individual units, said multiple linked individual units comprising: a bridging component selected from the group comprising a transition element, an inner transition element, a neighbor element of said transition element and a mixture of any of the foregoing elements, and a complexing component, provided that when said transition element is chromium, a chromium target molecule complex is created, further wherein said multiple linked individual units are combined with said supra-molecular lipid construct matrix.

7. The interferon binding supra-molecular lipid construct of claim 6, further comprising free interferon not associated with said target molecule complex.

8. The interferon binding supra-molecular lipid construct of claim 6, wherein said bridging component is chromium.

9. The interferon binding supra-molecular lipid construct of claim 6, wherein said complexing component comprises poly(bis)-[(N-(2,6-diisopropylphenyl)carbamoyl methyl) iminodiacetic acid].

10. The interferon binding supra-molecular lipid construct of claim 1, wherein the proximal moiety of the extended amphipathic lipid comprises at least one, but not more than two, long acyl hydrocarbon chains bound to a glycerol backbone, wherein said hydrocarbon chains may be saturated, unsaturated or a mixture thereof.

11. The interferon binding supra-molecular lipid construct of claim 1, wherein the medial moiety of the extended amphipathic lipid comprises a thio-acetyl triglycine polymer or a derivative thereof, wherein said amphipathic lipid molecule extends from the surface of the interferon binding supra-molecular lipid construct.

12. The interferon binding supra-molecular lipid construct of claim 1, wherein the distal component of the extended amphipathic lipid comprises a non-polar derivatized benzene ring or a heterobicyclic ring structure.

13. The interferon binding supra-molecular lipid construct of claim 1, wherein said construct comprises a positive charge or a negative charge or combinations thereof.

14. The interferon binding supra-molecular lipid construct of claim 1, wherein said extended amphipathic lipid molecule comprises at least one carbonyl moiety positioned at a distance approximately less than or equal to 13.5 angstroms from the terminal end of the distal moiety.

15. The interferon binding supra-molecular lipid construct of claim 1, wherein said extended amphipathic lipid molecule comprises at least one carbamoyl moiety comprising a secondary amine.

16. The interferon binding supra-molecular lipid construct of claim 1, wherein said extended amphipathic lipid molecule comprises positively charged chromium in said medial position.

17. The interferon binding supra-molecular lipid construct of claim 1 further comprising cellulose acetate hydrogen phthalate.

18. A method of manufacturing the interferon binding supra-molecular lipid construct of claim 1, comprising:
a. creating a mixture of the individual components of said lipid construct comprising amphipathic lipid molecules and an extended amphipathic lipid; and
b. forming a suspension of the target molecule complex in water.

19. The method of claim 18 further comprising the step of:
c. loading interferon into the supra-molecular lipid construct.

20. The method of claim 19, wherein said loading interferon into the supra-molecular lipid construct comprises equilibrium and non-equilibrium loading.

21. The method of claim 19, wherein the step of loading interferon into the supra-molecular lipid construct comprises adding interferon to a mixture of said supra-molecular lipid construct in water and allowing said interferon to remain in contact with said mixture until equilibrium to be reached.

22. The method of claim 21, further comprising the step of
d. terminally loading interferon into the supra-molecular lipid construct following said mixture reaching equilibrium, wherein the solution containing free interferon is removed from said construct, wherein said construct contains interferon bound to said construct.

23. The method of claim 18, further comprising the step of:
e. adding a chromium complex comprising multiple linked individual units to the supra-molecular lipid construct.

24. The method of claim 22, further comprising the step of:
f. adding cellulose acetate hydrogen phthalate to the supra-molecular lipid construct containing bound interferon.

25. A method of increasing the bioavailability of interferon in a patient comprising placing interferon within a supra-molecular lipid construct, wherein said supra-molecular lipid construct contains a plurality of non-covalent multi-dentate binding sites, said construct reducing the reactions of acids and enzymes in the stomach with said interferon and administering said interferon to said patient.

26. The method of claim 25, wherein said supra-molecular lipid construct comprises interferon, 1,2-distearoyl-sn-glycero-3-phophocholine, cholesterol, dicetyl phosphate, 1,2-dipalmitoyl-sn-glycero-[3-phospho-rac-(1-glycerol)], 1,2-distearoyl-sn-glycero-3-phosphoethanolamine, and 1,2-dipalmitoyl-sn-glycero-3-phosphoethanolamine-N-(succinyl) or derivatives, and a hepatocyte receptor binding molecule.

27. A method of treating a patient infected with hepatitis comprising administering to said patient an effective amount of a supra-molecular lipid construct comprising interferon associated with said construct.

28. The method of claim 27, wherein said hepatitis comprises at least one hepatitis selected from the group consisting of Hepatitis B, Hepatitis C, Hepatitis D, Hepatitis E, Hepatitis F and Hepatitis G.

29. The method of claim 27, wherein said supra-molecular lipid construct further comprises free interferon not associated with said target molecule complex.

30. The method of claim 27, wherein said supra-molecular lipid construct further comprises a target molecule complex, wherein said complex comprises multiple linked individual units.

31. The method of treating a patient according to claim 27, wherein said administration is oral or subcutaneous.

32. A method for increasing the delivery of interferon to hepatocytes in the liver of a patient infected with hepatitis by administering to said patient a supra-molecular lipid construct comprising interferon and an extended lipid molecule comprising a moiety that binds to hepatocyte receptors, wherein said supra-molecular lipid construct is present in a plurality of sizes, wherein hepatocyte receptors bind optimally sized constructs to augment endocytosis and elicit the intended pharmacological action of the supra-molecular lipid construct.

33. The method of claim 32, further comprising protecting said interferon molecule within said supra-molecular lipid construct from hydrolytic degradation by providing a shield of lipid molecules arranged in a three-dimensional structural array that prevents access by hydrolytic enzymes.

34. The method of claim 32, further comprising adding cellulose acetate hydrogen phthalate to the supra-molecular lipid construct to react with individual lipid molecules.

35. The method of claim 32, further comprising producing an insolubilized dosage form of said interferon within said supra-molecular lipid construct.

36. A kit for treating hepatitis in a mammal, said kit comprising interferon and interferon binding supra-molecular lipid construct, said kit further comprising physiological buffer solution, an applicator, and an instructional material for the use thereof.

37. The kit of claim 36, wherein said hepatitis comprises at least one hepatitis selected from the group consisting of Hepatitis B, Hepatitis C, Hepatitis D, Hepatitis E, Hepatitis F and Hepatitis G.
